(19) United States
(12) Patent Application Publication
(10) Pub. No.: US 2020/0294617 A1
(43) Pub. Date: Sep. 17, 2020

(54) A GRAPH-BASED CONSTANT-COLUMN BICLUSTERING DEVICE AND METHOD FOR MINING GROWTH PHENOTYPE DATA

(71) Applicant: KING ABDULLAH UNIVERSITY OF SCIENCE AND TECHNOLOGY, Thuwal (SA)

(72) Inventors: Xin GAO, Thuwal (SA); Majed Ateah ALZAHRANI, Thuwal (SA)

(21) Appl. No.: 16/644,693

(22) PCT Filed: Oct. 25, 2018
(86) PCT No.: PCT/IB2018/058332

§ 371 Date: Mar. 5, 2020

Related U.S. Application Data
(60) Provisional application No. 62/736,735, filed on Sep. 26, 2018.
(60) Provisional application No. 62/577,849, filed on Oct. 27, 2017.

(51) Int. Cl. G16B 20/00

(52) U.S. Cl. CPC G16B 20/00

(57) ABSTRACT

Provided is a device and method for detecting co-fit genes. The GRACOB device and method may detect co-fit genes from growth phenotype profiling data. The GRACOB device and method may discover the maximal constant-column biclusters, fully taking advantage of the properties of the growth phenotype profiling data. The identified co-fit genes may guide systems biology and synthetic biology studies and industries by determining important candidates for the growth of the respective microorganisms.

### CROSS REFERENCE TO RELATED APPLICATIONS

This application claims the benefit of U.S. Provisional Application No. 62/577,849 filed Oct. 27, 2017 and U.S. Provisional Application No. 62/736,735 filed Sep. 26, 2018, which are hereby incorporated by reference in their entirety.

### BACKGROUND

Under standard lab conditions, a vast majority of genes have little to no effect on the normal growth of microorganisms. These so-called “dispensable” genes account for over 90% in E. coli and B. subtilis, while over 80% in yeast. A molecular-network level understanding of the cause of this gene dispensability has important implications in evolution and systems biology.

Applicant has identified a number of deficiencies and problems associated with identifying these dispensable genes. Through applied effort, ingenuity, and innovation, many of these identified problems have been solved by developing solutions that are included in embodiments of the present invention, many examples of which are described in detail herein.

### BRIEF SUMMARY

In general, embodiments of the present invention provided herein include methods, devices, and computer program products for detecting co-fit genes. Provided herein is a device for detecting co-fit genes, the device comprising a processor and a memory storing computer instructions that, when executed by the processor, cause the device to transform genome-wide growth-phenotype data using a cumulative distribution function into transformed phenotype data disposed in a plurality of rows and columns. The device may sort the transformed phenotype data disposed in the plurality of columns independently of each column of the plurality of columns while retaining an original row index associated with each transformed phenotype data. The device may create a node for each set of consecutive rows in the plurality of rows. The device may create an edge between a pair of nodes in response to the pair of nodes being from different data columns sharing a number of consecutive rows over a row threshold. The device may delete any nodes having a number of consecutive rows under a column threshold. The device may determine maximal cliques from any remaining pairs of nodes, and the device may extract biclusters from the cliques to detect the co-fit genes.

In some embodiments, the plurality of columns may represent a plurality of stress conditions. In some embodiments, the plurality of rows may represent a plurality of strains.

In some embodiments, the nodes may be created for each set of consecutive rows in the plurality of rows such that the range of the transformed phenotype data in each consecutive row of the set of consecutive rows does not exceed a range threshold. In some embodiments, the range threshold may be a numerical range in which the transformed phenotype data of each consecutive row of the set of consecutive rows must fall. In some embodiments, the range threshold may be about 0.01 to about 0.10.

In some embodiments, the transformed phenotype data may be sorted in ascending order. In some embodiments, the memory storing computer instructions, when executed by the processor, may cause the device to repeat creation of an edge and deletion of any nodes.

In some embodiments, the row threshold may represent a number of strains or genes in each bicluster. In some embodiments, the column threshold may represent a number of stress conditions imposed on a strain or gene in the bicluster.

Embodiments provided herein are also directed to a method of detecting co-fit genes. The method may include transforming genome-wide growth-phenotype data using a cumulative distribution function into transformed phenotype data disposed in a plurality of rows and columns. The method may include sorting the transformed phenotype data disposed in the plurality of columns independently of each column of the plurality of columns while retaining an original row index associated with each transformed phenotype data. The method may include creating a node for each set of consecutive rows in the plurality of rows. The method may include creating an edge between a pair of nodes in response to the pair of nodes being from different data columns sharing a number of consecutive rows over a row threshold. The method may include deleting any nodes having a number of consecutive rows under a column threshold. The method may include determining maximal cliques from any remaining pairs of nodes. The method may include extracting biclusters from the cliques to detect the co-fit genes.

In some embodiments, the plurality of columns may represent a plurality of stress conditions. In some embodiments, the plurality of rows may represent a plurality of strains.

In some embodiments, the nodes may be created for each set of consecutive rows in the plurality of rows such that the range of the transformed phenotype data in each consecutive row of the set of consecutive rows does not exceed a range threshold. In some embodiments, the range threshold may be a numerical range in which the transformed phenotype data of each consecutive row of the set of consecutive rows must fall. In some embodiments, the range threshold may be about 0.01 to about 0.10. In some embodiments, the transformed phenotype data may be sorted in ascending order. In some embodiments, the method may include repeating the creation of an edge and deletion of any nodes.

In some embodiments, the row threshold may represent a number of strains or genes in each bicluster. In some embodiments, the column threshold may represent a number of stress conditions imposed on a strain or gene in the bicluster.

The foregoing brief summary is provided merely for purposes of summarizing some example embodiments illustrating some aspects of the present disclosure. Accordingly, it will be appreciated that the above-described embodiments are merely examples and should not be construed to narrow the scope of the present disclosure in any way. It will be appreciated that the scope of the present disclosure encompasses many potential embodiments in addition to those summarized herein, some of which will be described in further detail below.

### BRIEF DESCRIPTION OF THE FIGURES

Reference will now be made to the accompanying drawings, which are not necessarily drawn to scale, and wherein:

FIG. 1 illustrates a GRACOB system in accordance with some embodiments discussed herein;

FIG. 2 illustrates a schematic block diagram of circuitry that can be included in a GRACOB device in accordance with some embodiments discussed herein;

FIG. 3 illustrates an example GRACOB database in accordance with some embodiments discussed herein;

FIG. 4 illustrates example GRACOB circuitry in accordance with some embodiments discussed herein;

FIG. 5a illustrates environment-dependent genetic interactions in accordance with some embodiments discussed herein;

FIG. 5b illustrates the corresponding growth phenotype data in accordance with some embodiments discussed herein;

FIGS. 6a and 6b illustrate a flow diagram of exemplary operations of a GRACOB device or system in accordance with some embodiments discussed herein;

FIG. 7 parts 1-12d provide a heatmap visualization of the E. coli growth phenotype data and the representative biclusters detected by 11 methods;

FIGS. 8a-8l provide a performance comparison of the 11 methods on the E. coli, proteobacteria, and yeast growth phenotype datasets;

FIG. 9 parts 1a-8d illustrates the performance comparison on the synthetic data sets;

FIGS. 10a-10d show the GO term enrichment precision under different significance levels for the three branches of the GO hierarchy for E. coli, proteobacteria, and yeast, respectively;

FIG. 11 illustrates a parameter sensitivity analysis for the GRACOB device and method in terms of the KEGG pathway-level precision of the detected biclusters on the E. coli data set in accordance with some embodiments discussed herein;

FIG. 12 illustrates a parameter sensitivity analysis for the GRACOB device and method in terms of the GO term-level precision of the detected biclusters on the E. coli data set in accordance with some embodiments discussed herein;

FIG. 13 illustrates a pathway map of genes from the case study bicluster as shown in FIG. 7 part (11a) in accordance with some embodiments discussed herein;

FIG. 14 illustrates a heatmap of a bicluster determined by the GRACOB device and method in accordance with some embodiments discussed herein; and

FIG. 15 illustrates a sample bicluster of size 11×5 with mixed colors that illustrate a grouping of genes based on both conditional essentiality and dispensability criteria.

### DETAILED DESCRIPTION

Various embodiments of the inventions now will be described more fully hereinafter, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level.

As used herein, the terms “data,” “content,” “digital content,” “digital content object,” “information,” and similar terms may be used interchangeably to refer to data capable of being transmitted, received, and/or stored in accordance with embodiments of the present invention. Thus, use of any such terms should not be taken to limit the spirit and scope of embodiments of the present invention. Further, where a computing device is described herein to receive data from another computing device, it will be appreciated that the data may be received directly from the another computing device or may be received indirectly via one or more intermediary computing devices, such as, for example, one or more servers, relays, routers, network access points, base stations, hosts, and/or the like, sometimes referred to herein as a “network.” Similarly, where a computing device is described herein to send data to another computing device, it will be appreciated that the data may be sent directly to the another computing device or may be sent indirectly via one or more intermediary computing devices, such as, for example, one or more servers, relays, routers, network access points, base stations, hosts, and/or the like.

The term “client device” refers to computer hardware and/or software that is configured to access a service made available by a server. The server is often (but not always) on another computer system, in which case the client device accesses the service by way of a network. Client devices may include, without limitation, smart phones, tablet computers, laptop computers, wearables, personal computers, enterprise computers, and the like.

The term “user” should be understood to refer to an individual, group of individuals, business, organization, and the like; the users referred to herein are accessing the GRACOB system using client devices.

### Overview

Provided herein are systems, methods, devices, and computer program products to detect co-fit genes.

As previously discussed herein, there exists a vast majority of genes have little to no effect on the normal growth of microorganisms, also calls “dispensable” genes. One theory to explain this phenomenon is mutational robustness, which argues that these genes are dispensable because the genetic architecture has evolved to compensate for gene mutations either by duplicate genes or by backup pathways. Another theory is environment-dependent genetic interaction, which argues that these seemingly dispensable genes are actually essential in other environments as the activation of genetic interactions depends on environmental conditions. Whereas both theories could explain dispensable genes, the latter was shown to provide explanations for a majority of dispensable genes in yeast. To advance knowledge of environment-dependent genetic interactions, one key question to address is how to find co-fit genes, which are defined to be a group of genes that share similar patterns of conditional essentiality and dispensability across various environmental conditions.

The recent development in genome-wide growth-phenotype (i.e. fitness) profiling methods enabled the measurement of fitness scores of a large number of gene-deletion strains over many stress conditions. Importantly, such growth phenotype data can be used to assess the effects of a loss-of-function mutation of each gene on fitness and detect which genes are essential and dispensable under different stress conditions. That is, for a given environmental condition, conditionally essential genes are defined to be those whose loss-of-function mutations have very low fitness values, while conditionally dispensable genes are defined to be those whose loss-of-function mutations have very high fitness values. Thus, such growth phenotype data can be used to systematically identify sets of co-fit genes, allowing probing into how the genetic interactions are organized and how environmental conditions can change the genetic interactions. Such environment-dependent genetic interactions have been commonly analyzed using flux balance analysis. While flux balance analysis may be a powerful method that can predict how metabolic activities may change given various environmental and genetic perturbations, its accuracy depends on prior knowledge about the structure of a given metabolic system and metabolic flux boundaries.

There exists a need for an alternative, data-driven approach that can be used for analysis of environment-dependent genetic interactions. In the presently disclosed devices and methods, referred to herein as the GRACOB (graph-based constant-column biclustering) system, device, and method, according to certain embodiments described herein, by representing a growth phenotype data set by a two-dimensional matrix, whose rows are the gene-deletion strains and columns are the stress conditions, the problem of finding sets of co-fit genes is transformed into a constant-column biclustering problem.

In particular, in growth phenotype data, finding constant-column biclusters results in detecting more meaningful biclusters, i.e. co-fit genes. There is a fundamental difference in the nature of growth phenotype data and gene expression data. In gene expression data, each row (i.e. a gene) has a reference value, which is the expression level of this gene under the normal condition. Thus, the reference values for different rows are different from each other. Although data normalization or transpose may be done to transform the problem of mining gene expression data into the constant-column biclustering problem, mining other types of biclusters, e.g., constant biclusters or coherent biclusters, is more prevalent in mining gene expression data. In contrast, in growth phenotype data, all rows (i.e. strains) have the same reference value, which is the growth of the wild type (without any knock-out) under the normal condition. Thus, detecting constant-column biclusters in such data can identify co-fit genes because such a bicluster implies the deletion of this group of genes has similar effects on fitness (i.e. similar values in the same column imply similar changes to the reference value) under a subset of stress conditions.

Certain embodiments discussed herein include a biclustering device and method, which are designed to identify constant-column biclusters in growth phenotype data sets. In particular, the GRACOB device, system, and method discussed herein develops and applies biclustering methods to mining co-fit genes in growth phenotype data. The identification of co-fit genes by the GRACOB device, system, and method can be useful for gaining new insights into the functional organization of genes. This is because a co-fit gene measure can detect a significant local fitness similarity under a subset of conditions, while such strong signals can be diluted in the overall correlation coefficient measure owing to the rest of the conditions.

Growth phenotype profiling of genome-wide gene-deletion strains over stress conditions can offer a clear picture that the essentiality of genes depends on environmental conditions. Systematically identifying groups of genes from such high-throughput data that share similar patterns of conditional essentiality and dispensability under various environmental conditions can elucidate how genetic interactions of the growth phenotype are regulated in response to the environment.

Detecting such ‘co-fit’ gene groups can be cast as a less well-studied problem in biclustering, i.e. constant-column biclustering. Despite significant advances in biclustering techniques, very few were designed for mining in growth phenotype data. The present device and method provide an efficient graph-based method that casts and solves the constant-column biclustering problem as a maximal clique finding problem in a multipartite graph. The present device and method was compared with a large collection of other biclustering methods that cover different types of methods designed to detect different types of biclusters. The present device and method showed superior performance on finding co-fit genes over all the existing methods on both a variety of synthetic data sets with a wide range of settings, and three real growth phenotype datasets for E. coli, proteobacteria and yeast.

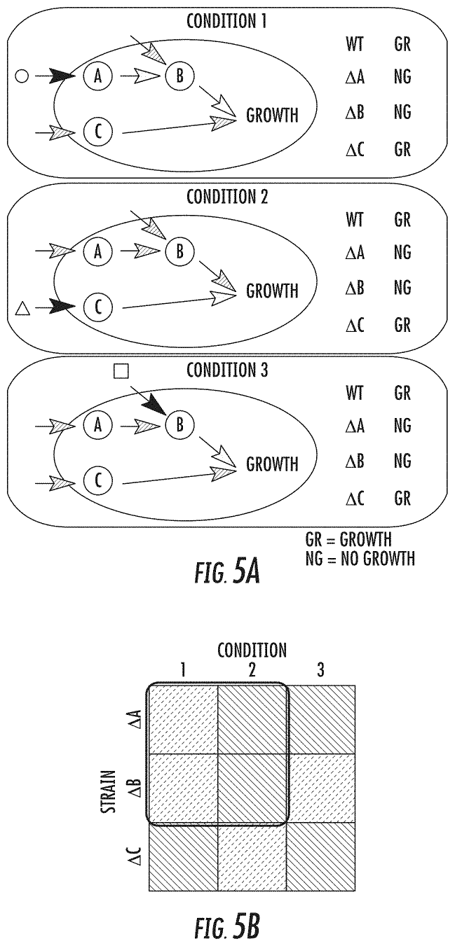

FIG. 5a-5b illustrates how similar phenotype patterns can help reveal the underlying organization of the genetic interactions. FIG. 5a shows environment-dependent genetic interactions. The circle, triangle and square symbols illustrate environmental inputs to the cell, for example, input metabolites and ligands. White, striped, and black arrows denote active paths in the wild type, inactive paths, and active paths, respectively, in each condition. The wild type grows normally under each condition, while the deletion of each gene has different effects on fitness under different conditions. ΔX denotes the strain of deleting gene X (X∈{A,B,C}). “GR” and “NG” stand for normal growth and no growth, respectively. FIG. 5b illustrates the corresponding growth phenotype data. Dots and stripes denote low and high fitness, respectively. The constant-column bicluster in the outlined box captures co-fit genes, A and B, which cannot be captured by any other constant biclusters.

When evaluated on a variety of synthetic data sets, the GRACOB system, device, and method may show nearly perfect performance with respect to different noise levels and overlapping degrees. The GRACOB system, device, and method were then applied to three real growth phenotype data sets for E. coli, proteobacteria, and yeast, and was able to identify maximal constant-column biclusters while prior existing methods failed to do so. Functional enrichment analysis through KEGG pathways and GO terms demonstrated that the GRACOB device and method may be on average more than twice as precise as other methods.

Existing methods mainly deal with three types of biclusters, i.e. constant biclusters within which the variation is low, constant-column (or constant-row) biclusters within which the column-wise (or the row-wise) variation is low, and coherent biclusters in which the data generally follow an additive or a multiplicative model. The GRACOB system, device, and method may determine a group of genes that, under multiple conditions, have similar fitness to each other.

By way of review, 13 biclustering methods that are widely used in various comparative studies are discussed herein. As further discussed below, these method were compared with various aspects of the presently disclosed system, device, and method on both synthetic datasets and real growth phenotype datasets. These methods are CC (Cheng and Church, 2000), Plaid (Lazzeroni and Owen, 2002; Turner et al., 2005), FLOC (Yang et al., 2003), ISA (Bergmann et al., 2003), xMOTIFs (Murali and Kasif, 2003), Spectral (Kluger et al., 2003), SAMBA (Tanay et al., 2004), Bimax (Prelić et al., 2006), BBC (Gu and Liu, 2008), QUBIC (Li et al., 2009), CPB (Bozda\check{g} et al., 2009), iBBiG (Gusenleitner et al., 2012) and BicPAM (Henriques and Madeira, 2014). Since most of the existing methods used different definitions of biclusters and were reported to be general as they are not restricted to certain types of data, it is difficult to clearly categorize them.

The biclustering methods can generally be grouped according to the general types of biclusters such methods used for evaluation in their papers or in comparative studies. A typical class of the existing methods work with “constant” biclusters. Here constant is often defined to be the same value after discretizing the input data matrix into 0's and 1's (e.g. Bimax and iBBiG).

Another major class of the existing methods have their own definitions of the biclusters they are looking for, which do not directly correspond to constant-, constant-column-, or coherent-biclusters. For example, CC uses the mean squared residue to define a bicluster, which basically measures the variance of the individual data points in the biclusters with respect to the mean of the corresponding rows, the corresponding columns, and the entire bicluster. Plaid models the data matrix as a sum of layers and minimizes the fitting error through optimization. Similarly, BBC uses the plaid model of biclusters which defines a bicluster as a combination of the main effect, the gene effect, the condition effect, and the noise. FLOC extends the CC model by using a probabilistic model to account for missing values in data.

ISA requests that the mean value of each row must be higher than a threshold, and so does each column CPB defines the biclusters in a similar way, i.e. the Pearson correlation coefficient between columns and rows must be higher than a threshold. Spectral tries to detect checkerboard structures. Therefore, this class of methods can theoretically detect different types of biclusters.

A number of methods were developed to (preferably) detect constant-column (or equivalently constant-row) biclusters. SAMBA discretizes the data into different bins and finds biclusters with each column belonging to the same bin. Similarly, xMOTIFs attempts to find biclusters within each of which genes have the same state under different samples. The method picks up randomly sampled subsets over the conditions and chooses the corresponding subsets of genes that satisfy this requirement. However, when the number of conditions is large, the chance of picking the proper subsets of conditions becomes very low. QUBIC thresholds the extreme values (both positive and negative) and detects constant-column and constant-row biclusters on the discretized values only. Recently, BicPAM was proposed to detect both additive and multiplicative coherent biclusters.

In terms of the techniques such methods use, they can be classified into iterative methods (i.e. CC, ISA, Bimax, CPB, Plaid, FLOC and iBBiG), matrix decomposition-based methods (i.e. ISA and Spectral), graph-based methods (i.e. SAMBA and QUBIC), sampling-based methods (i.e. xMOTIFs and BBC) and pattern mining-based methods (i.e. BicPAM). The iterative methods either gradually grow biclusters from small seeds, or delete columns or rows that cannot be a part of the biclusters from the original matrix. The decomposition-based methods mainly use different variants of singular value decomposition to reduce the dimensionality in order to better detect biclusters. The graph-based methods model the problem in a bipartite graph and look for cliques or densely connected subgraphs. The sampling-based methods try to control the way of sampling to increase the probability of finding large biclusters. The pattern mining-based methods rely on frequent itemset mining or association rules to identify biclusters.

Co-fit genes may be defined using the pairwise correlation coefficient of two genes across all the stress conditions, and hierarchical clustering may be used to group co-fit genes together. However, the use of correlation coefficients to measure similarity could miss strong signals detected in a subset of conditions owing to “correlation dilution” through the rest of the conditions. For example, the genes LSM2 and LSM3 of Saccharomyces cerevisiae have a low correlation value, r=0.15, although the genes share many common functions and high sequence similarity. Both genes are part of one complex that binds to the 3′ end of U6 snRNA, and are responsible for its regulation and stability. LSM2 and LSM3 are required for pre-mRNA splicing and the genes' mutations inhibit mRNA decapping. LSM2 and LSM3 form many interactions with each other. The semantic similarity between their cellular component GO terms is 0.95 as calculated using Wang et al. (2007). Thus, these two genes are in the same functional organization by definition. However, the correlation coefficient measurement cannot capture this. In contrast, the GRACOB system, device, and method may predict the genes as co-fit genes since the genes were in the same constant-column bicluster based on similar fitness values representing conditional essentiality or dispensability. Specifically, the GRACOB system, device, and method detected similar, extreme fitness values between the LSM2- and LSM3-deletion strains for 51 out of 726 different stress conditions in the yeast phenotype profiling data showing statistically significant association (e.g., P-value=3.0×10⁻⁶). These deletion strains have a very high correlation (r=0.99) over these 51 conditions.

Using the GRACOB system, device, and method, co-fitness may be detected by local measures to capture the similarity over a subset of conditions. Furthermore, by using the GRACOB system, device, and method to find co-fit genes, it may be possible to explicitly identify which subset of genes shares similar patterns of conditional essentiality and dispensability under which subset of stress conditions. By definition of co-fitness, a bicluster of co-fit genes should have similar values in each column of this bicluster, but values across different columns may be very different.

### Example System Architecture

Methods, systems, devices, and computer program products of the present disclosure may be embodied by any of a variety of devices. For example, the method, system, device, and computer program product of an example embodiment may be embodied by a networked device (e.g., an enterprise platform), such as a server or other network entity, configured to communicate with one or more devices, such as one or more client devices. Additionally or alternatively, the computing device may include fixed computing devices, such as a personal computer or a computer workstation. Still further, example embodiments may be embodied by any of a variety of mobile devices, such as a portable digital assistant (PDA), mobile telephone, smartphone, laptop computer, tablet computer, wearable, or any combination of the aforementioned devices.

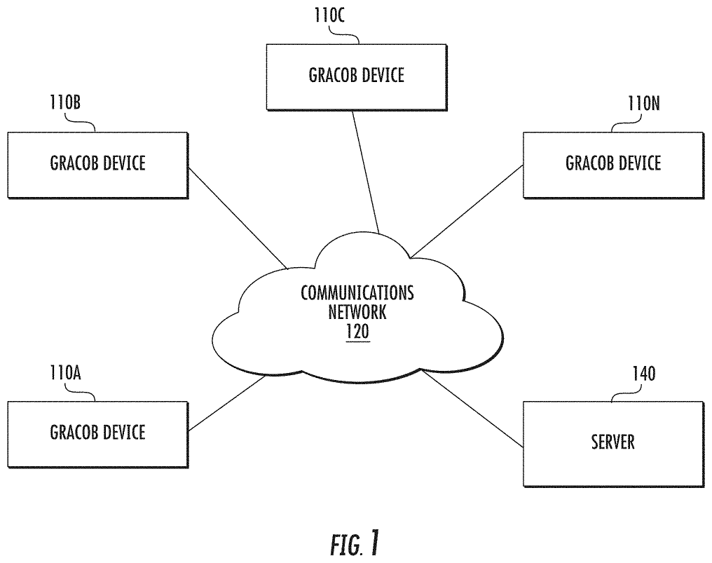

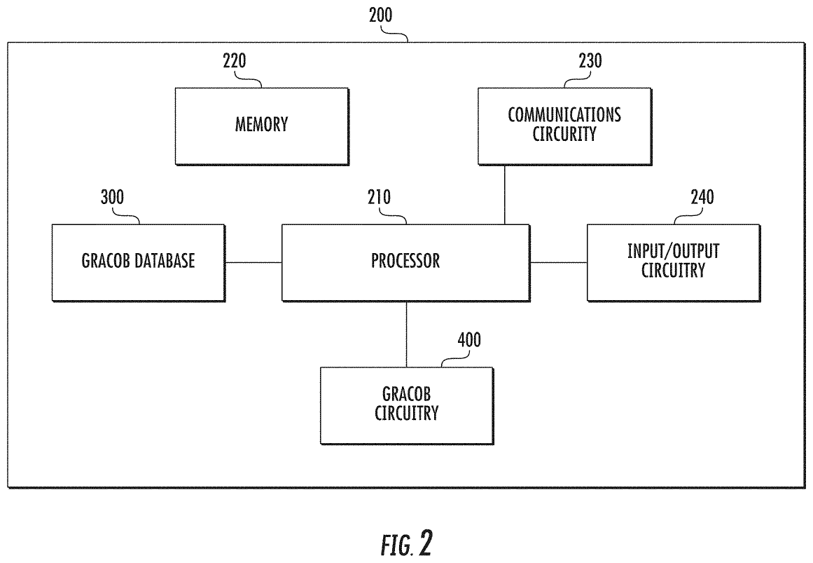

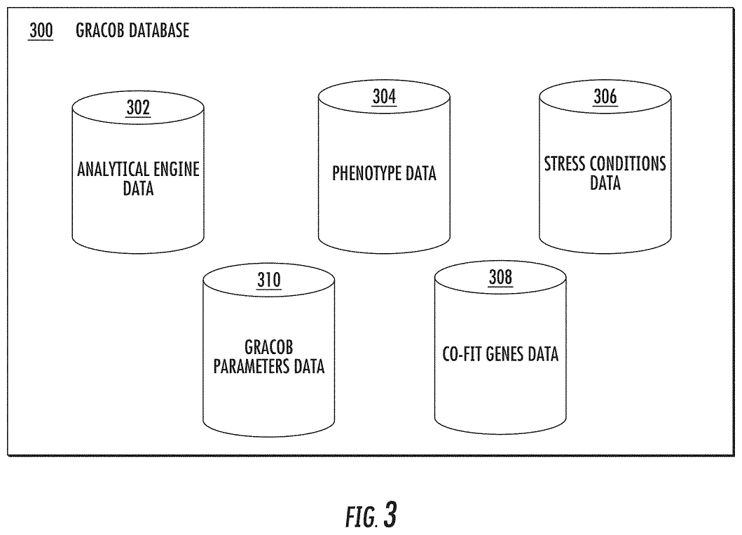

FIG. 1 shows GRACOB system 100 including an example network architecture for a system, which may include one or more devices and sub-systems that are configured to implement some embodiments discussed herein. For example, GRACOB system 100 may include server 140, which can include, for example, the circuitry disclosed in FIGS. 2-3B, a server, or database, among other things (not shown). The server 140 may include any suitable network server and/or other type of processing device. In some embodiments, the server 140 may determine and transmit commands and instructions for determining co-fit genes to GRACOB devices 110A-110N using data from the GRACOB database 300. The GRACOB database 300 may be embodied as a data storage device such as a Network Attached Storage (NAS) device or devices, or as a separate database server or servers. The GRACOB database 300 includes information accessed and stored by the server 140 to facilitate the operations of the GRACOB system 100. For example, the GRACOB database 300 may include, without limitation, a plurality of genes, stress conditions, phenotypes, and/or the like.

Server 140 can communicate with one or more GRACOB devices 110A-110N via network 120. In this regard, network 120 may include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, etc.). For example, communications network 120 may include a cellular telephone, an 802.11, 802.16, 802.20, and/or WiMax network. Further, the communications network 120 may include a public network, such as the Internet, a private network, such as an intranet, or combinations thereof, and may utilize a variety of networking protocols now available or later developed including, but not limited to TCP/IP based networking protocols. For instance, the networking protocol may be customized to suit the needs of the GRACOB system, device, and method.

The server 140 may provide for receiving of electronic data from various sources, including but not necessarily limited to the GRACOB devices 110A-110N. For example, the server 140 may be operable to receive, transmit, store, or analyze various data and inputs provided by the GRACOB devices 110A-110N.

GRACOB devices 110A-110N and/or server 140 may each be implemented as a personal computer and/or other networked device, such as a cellular phone, tablet computer, mobile device, etc., that may be used for any suitable purpose. The depiction in FIG. 1 of “N” users is merely for illustration purposes. Any number of users may be included in the GRACOB system 100. In one embodiment, the GRACOB devices 110A-110N may be configured to view, create, edit, and/or otherwise interact with co-fit gene data and other data discussed herein, which may be provided by the server 140. According to some embodiments, the server 140 may be configured to view, create, edit, and/or otherwise interact with co-fit gene data and other data discussed herein. In some embodiments, an interface of a GRACOB device 110A-110N may be different from an interface of a server 140. The GRACOB devices 110A-110N may be used in addition to or instead of the server 140. GRACOB system 100 may also include additional client devices and/or servers, among other things. Additionally or alternatively, the GRACOB device 110A-110N may interact with the GRACOB system 100 via a web browser. As yet another example, the GRACOB device 110A-110N may include various hardware or firmware designed to interface with the GRACOB system 100.

The GRACOB devices 110A-110N may be any computing device as defined above. Electronic data received by the server 140 from the GRACOB devices 110A-110N may be provided in various forms and via various methods. For example, the GRACOB devices 110A-110N may include desktop computers, laptop computers, smartphones, netbooks, tablet computers, wearables, and the like.

In embodiments where a GRACOB device 110A-110N is a mobile device, such as a smart phone or tablet, the GRACOB device 110A-110N may execute an “app” to interact with the GRACOB system 100. Such apps are typically designed to execute on mobile devices, such as tablets or smartphones. For example, an app may be provided that executes on mobile device operating systems such as iOS®, Android®, or Windows®. These platforms typically provide frameworks that allow apps to communicate with one another and with particular hardware and software components of mobile devices. For example, the mobile operating systems named above each provide frameworks for interacting with location services circuitry, wired and wireless network interfaces, user contacts, and other applications. Communication with hardware and software modules executing outside of the app is typically provided via application programming interfaces (APIs) provided by the mobile device operating system. Communications may be sent over communications network 120 directly by a GRACOB device 110A-110N or via an intermediary such as a message server, and/or the like. For example, the GRACOB device 110A-110N may be a desktop, a laptop, a tablet, a smartphone, and/or the like that is executing a client application (e.g., an app).

The GRACOB system 100 may comprise at least one server 140 that may create a storage communication based upon the received data to facilitate indexing and storage in a database, as will be described further below. In one implementation, the communications/data may be parsed (e.g., using PHP commands) to determine context for the message. FIG. 2 shows a schematic block diagram of an apparatus 200, some or all of the components of which may be included, in various embodiments, in one or more devices. Any number of systems or devices may include the components of apparatus 200 and may be configured to, either independently or jointly with other devices to perform the functionality of the apparatus 200 described herein resulting in a GRACOB system or device. As illustrated in

FIG. 2, in accordance with some example embodiments, apparatus 200 can includes various means, such as processor 210, memory 220, communications circuitry 230, and/or input/output circuitry 240. In some embodiments, GRACOB database 300 and/or GRACOB circuitry 400 may also or instead be included. As referred to herein, “circuitry” includes hardware, or a combination of hardware with software configured to perform one or more particular functions. In this regard, the various components of apparatus 200 described herein may be embodied as, for example, circuitry, hardware elements (e.g., a suitably programmed processor, combinational logic circuit, and/or the like), a computer program product comprising computer-readable program instructions stored on a non-transitory computer-readable medium (e.g., memory 220) that is executable by a suitably configured processing device (e.g., processor 210), or some combination thereof. In some embodiments, one or more of these circuitries may be hosted remotely (e.g., by one or more separate devices or one or more cloud servers) and thus need not reside on the data set device or user device. The functionality of one or more of these circuitries may be distributed across multiple computers across a network.

Processor 210 may, for example, be embodied as various means including one or more microprocessors with accompanying digital signal processor(s), one or more processor(s) without an accompanying digital signal processor, one or more coprocessors, one or more multi-core processors, one or more controllers, processing circuitry, one or more computers, various other processing elements including integrated circuits such as, for example, an ASIC (application specific integrated circuit) or FPGA (field programmable gate array), or some combination thereof. Accordingly, although illustrated in FIG. 2 as a single processor, in some embodiments processor 210 comprises a plurality of processors. The plurality of processors may be embodied on a single computing device or may be distributed across a plurality of computing devices collectively configured to function as apparatus 200. The plurality of processors may be in operative communication with each other and may be collectively configured to perform one or more functionalities of apparatus 200 as described herein. In an example embodiment, processor 210 is configured to execute instructions stored in memory 220 or otherwise accessible to processor 210. These instructions, when executed by processor 210, may cause apparatus 200 to perform one or more of the functionalities as described herein.

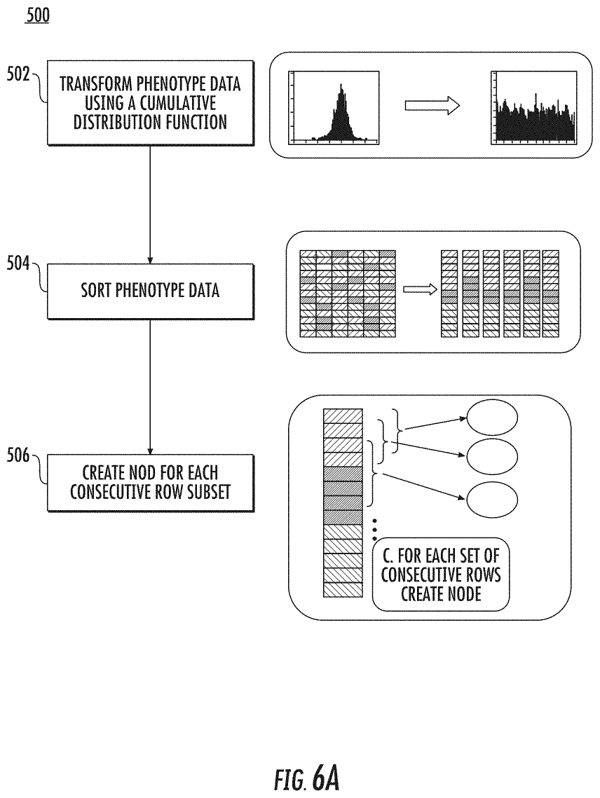

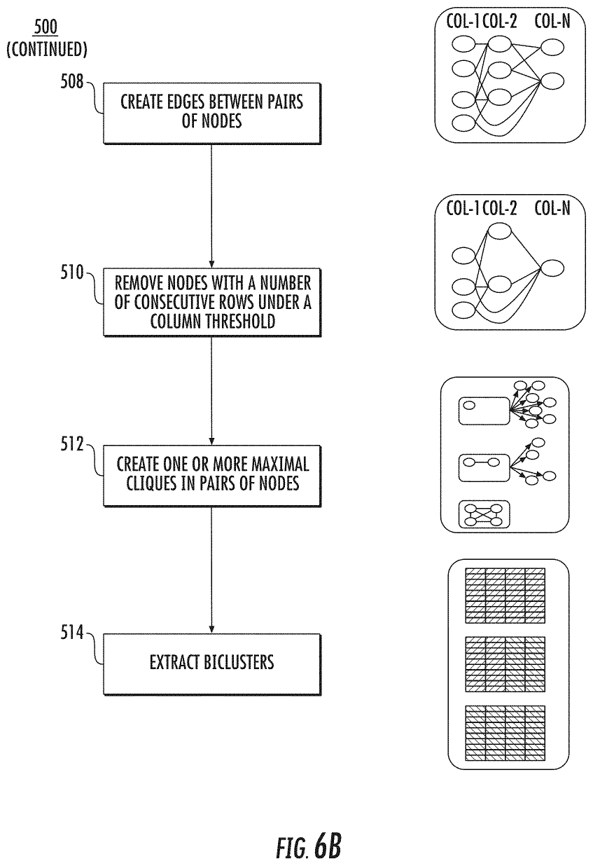

Whether configured by hardware, or a combination of hardware with firmware/software methods, processor 210 may comprise an entity capable of performing operations according to embodiments of the present invention while configured accordingly. Thus, for example, when processor 210 is embodied as an ASIC, FPGA or the like, processor 210 may comprise the specifically configured hardware for conducting one or more operations described herein. Alternatively, as another example, when processor 210 is embodied as an executor of instructions, such as may be stored in memory 220, the instructions may specifically configure processor 210 to perform one or more algorithms and operations described herein, such as those discussed in connection with FIGS. 6a-6b.

Memory 220 may comprise, for example, volatile memory, non-volatile memory, or some combination thereof. Although illustrated in FIG. 2 as a single memory, memory 220 may comprise a plurality of memory components. The plurality of memory components may be embodied on a single computing device or distributed across a plurality of computing devices. In various embodiments, memory 220 may comprise, for example, a hard disk, random access memory, cache memory, flash memory, a compact disc read only memory (CD-ROM), digital versatile disc read only memory (DVD-ROM), an optical disc, circuitry configured to store information, or some combination thereof. Memory 220 may be configured to store information, data (including item data and/or profile data), applications, instructions, or the like for enabling apparatus 200 to carry out various functions in accordance with example embodiments of the present invention. For example, in at least some embodiments, memory 220 is configured to buffer input data for processing by processor 210. Additionally or alternatively, in at least some embodiments, memory 220 is configured to store program instructions for execution by processor 210. Memory 220 may store information in the form of static and/or dynamic information. This stored information may be stored and/or used by apparatus 200 during the course of performing its functionalities.

Communications circuitry 230 may be embodied as any device or means embodied in circuitry, hardware, a computer program product comprising computer readable program instructions stored on a computer readable medium (e.g., memory 220) and executed by a processing device (e.g., processor 210), or a combination thereof that is configured to receive and/or transmit data from/to another device and/or network, such as, for example, a second apparatus 200 and/or the like. In some embodiments, communications circuitry 230 (like other components discussed herein) can be at least partially embodied as or otherwise controlled by processor 210. In this regard, communications circuitry 230 may be in communication with processor 210, such as via a bus. Communications circuitry 230 may include, for example, an antenna, a transmitter, a receiver, a transceiver, network interface card and/or supporting hardware and/or firmware/software for enabling communications with another computing device. Communications circuitry 230 may be configured to receive and/or transmit any data that may be stored by memory 220 using any protocol that may be used for communications between computing devices. Communications circuitry 230 may additionally or alternatively be in communication with the memory 220, input/output circuitry 240 and/or any other component of apparatus 200, such as via a bus.

Input/output circuitry 240 may be in communication with processor 210 to receive an indication of a user input and/or to provide an audible, visual, mechanical, or other output to a user (e.g., provider and/or consumer). Some example visual outputs that may be provided to a user by apparatus 200 are discussed in connection with FIGS. 6a-6b. As such, input/output circuitry 240 may include support, for example, for a keyboard, a mouse, a joystick, a display, a touch screen display, a microphone, a speaker, a RFID reader, barcode reader, biometric scanner, and/or other input/output mechanisms. In embodiments wherein apparatus 200 is embodied as a server or database, aspects of input/output circuitry 240 may be reduced as compared to embodiments where apparatus 200 is implemented as an end-user machine (e.g., lab payer device and/or provider device) or other type of device designed for complex user interactions. In some embodiments (like other components discussed herein), input/output circuitry 240 may even be eliminated from apparatus 200. Alternatively, such as in embodiments wherein apparatus 200 is embodied as a server or database, at least some aspects of input/output circuitry 240 may be embodied on an apparatus used by a user that is in communication with apparatus 200. Input/output circuitry 240 may be in communication with the memory 220, communications circuitry 230, and/or any other component(s), such as via a bus. One or more than one input/output circuitry and/or other component can be included in apparatus 200.

GRACOB database 300 and GRACOB circuitry 400 may also or instead be included and configured to perform the functionality discussed herein related to storing, generating, and/or editing data. In some embodiments, some or all of the functionality of these components of the apparatus 200 may be performed by processor 210, although in some embodiments, these components may include distinct hardware circuitry designed to perform their respective functions. In this regard, the example processes and algorithms discussed herein can be performed by at least one processor 210, GRACOB database 300, and/or GRACOB circuitry 400. For example, non-transitory computer readable media can be configured to store firmware, one or more application programs, and/or other software, which include instructions and other computer-readable program code portions that can be executed to control each processor (e.g., processor 210, GRACOB database 300, and GRACOB circuitry 400) of the components of apparatus 200 to implement various operations, including the examples shown above. As such, a series of computer-readable program code portions are embodied in one or more computer program goods and can be used, with a computing device, server, and/or other programmable apparatus, to produce machine-implemented processes.

In some embodiments, the GRACOB database 300 (see FIG. 3) may store phenotype data 304, stress conditions data 306, co-fit genes data 308, GRACOB parameters data 310, and/or analytical engine data 302. Phenotype data 304 may be organized by strain and may include various information associated with a phenotype or strain in the phenotype data 304. Stress conditions data 306 may include various conditions, such as temperature, pH, salt content, etc. and may be associated with phenotype data. Co-fit genes data 308 may include various co-fit genes and may include various information associated with the co-fit genes. GRACOB parameters data 310 may include the parameters c, r, and δ, where c is the column threshold, r is the row threshold, and δ is the range threshold. These parameters will be discussed in more detail below. The various data may be retrieved from any of a variety of sources, such as any device that may interact with the GRACOB system 100.

Additionally or alternatively, the GRACOB database 300 may include analytical engine data 302 which provides any additional information needed by the processor 210 in analyzing and generating data.

Overlap among the data obtained by the GRACOB database 300 among the phenotype data 304, stress conditions data 306, co-fit genes data 308, GRACOB parameter data 310, and/or analytical engine data 302 may occur and information from one or more of these databases may be retrieved from any device that may interact with the GRACOB system 100, such as a client device operated by a user. As new data is obtained by the apparatus 200, such data may be retained in the GRACOB database 300 in one or more of the phenotype data 304, stress conditions data 306, co-fit genes data 308, GRACOB parameter data 310, and analytical engine data 302.

GRACOB circuitry 400 can be configured to analyze multiple sets of GRACOB parameters, phenotype data, and stress conditions as discussed herein and combinations thereof, such as any combination of the data in the GRACOB database 300, to determine co-fit genes. In this way, GRACOB circuitry 400 may execute multiple algorithms, including those discussed below with respect to the GRACOB system 100.

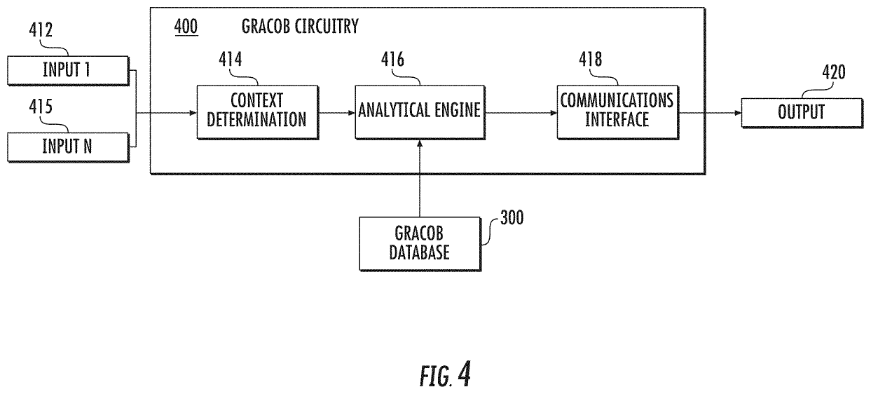

In some embodiments, with reference to FIG. 4, the GRACOB circuitry 400 may include a context determination module 414, an analytical engine 416, and communications interface 418, all of which may be in communication with the GRACOB database 300. In some embodiments, the context determination module 414 may be implemented using one or more of the components of apparatus 200. For instance, the context determination module 414 may be implemented using one or more of the processor 210, memory 220, communications circuitry 230, and input/output circuitry 240. For instance, the context determination module 414 may be implemented using one or more of the processor 210 and memory 220. The analytical engine 416 may be implemented using one or more of the processor 210, memory 220, communications circuitry 230, and input/output circuitry 240. For instance, the analytical engine 416 may be implemented using one or more of the processor 210 and memory 220. The communications interface 418 may be implemented using one or more of the processor 210, memory 220, communications circuitry 230, and input/output circuitry 240. For instance, the communications interface 418 may be implemented using one or more of the communications circuitry 230 and input/output circuitry 240.

The GRACOB circuitry 400 may receive one or more GRACOB parameters, phenotype data, and stress conditions and may generate the appropriate response as will be discussed herein (see e.g., FIGS. 6a-6b). The GRACOB circuitry 400 may use any of the algorithms or processes disclosed herein for receiving any of the GRACOB parameters, phenotype data, and stress conditions, etc. discussed herein and generating the appropriate response. In some other embodiments, such as when the apparatus 200 is embodied in a server and/or client devices, the GRACOB circuitry 400 may be located in another apparatus 200 or another device, such as another server and/or client devices.

The GRACOB system 100 may receive a plurality of inputs 412, 415 from the apparatus 200 and process the inputs within the GRACOB circuitry 400 to produce an output 420, which may include appropriate transformed phenotype data, sorted transformed phenotype data, nodes, edges, maximal cliques, biclusters, etc. in response. In some embodiments, the GRACOB circuitry 400 may execute context determination using the context determination module 414, process the communication and/or data in an analytical engine 416, and output the results via a communications interface 418. Each of these steps may retrieve data from a variety of sources including the GRACOB database 300.

When inputs 412, 415 are received by the GRACOB circuitry 400, the context determination module 414 may make a context determination regarding the communication. A context determination includes such information as when and what user initiated generation of the input (e.g., when and who selected the actuator that initiated the transformation), what type of input was provided (e.g., phenotype data or stress conditions) and under what circumstances receipt of the input was initiated (e.g., GRACOB parameters). This information may give context to the GRACOB circuitry 400 analysis for subsequent determinations. For example, the context determination module 414 may inform the GRACOB circuitry 400 as to the content to output.

The GRACOB circuitry 400 may then compute the output using the analytical engine 416. The analytical engine 416 draws the applicable data from the GRACOB database 300 and then, based on the context determination made by the context determination module 414, computes an output, which may vary based on the input. The communications interface 418 then outputs the output 420 to the apparatus 200 for display on the appropriate device. For instance, the context determination module 414 may determine that certain phenotype data or GRACOB parameters were obtained. Based on this information as well as the applicable data from the GRACOB database 300 (e.g., additional phenotype data, GRACOB parameter data, stress conditions data, co-fit genes data, etc.), the analytical engine 416 may determine an appropriate output 420, such as transformed phenotype data, sorted transformed phenotype data, nodes, edges, maximal cliques, biclusters, co-fit genes, etc. The analytical engine 416 may also determine that certain data in the GRACOB database 300 should be updated to reflect the new information contained in the received input.

In some embodiments of an exemplary system, GRACOB parameters data, phenotype data, stress conditions data, etc. may be sent from a user (via a client device) to apparatus 200. In various implementations, GRACOB parameters data, phenotype data, stress conditions data, etc. may be sent directly to the apparatus 200 (e.g., via a peer-to-peer connection) or over a network, in which case the GRACOB parameters data, phenotype data, stress conditions data, co-fit genes data, etc. may in some embodiments be transmitted via an intermediary such as a message server, and/or the like.

In one implementation, the GRACOB parameters data, phenotype data, stress conditions data, etc. may be parsed by the apparatus 200 to identify various components included therein. Parsing of the GRACOB parameters data, phenotype data, stress conditions data, co-fit gene data, etc. may facilitate determination by the apparatus 200 of the user who sent the information and/or to the contents of the information and to what or whom the information relates. Machine learning techniques may be used.

In embodiments, the contents of the GRACOB parameters data, phenotype data, stress conditions data, co-fit genes data, etc. may be used to index the respective information to facilitate various facets of searching (i.e., search queries that return results from GRACOB database 300).

As will be appreciated, any such computer program instructions and/or other type of code may be loaded onto a computer, processor or other programmable apparatus's circuitry to produce a machine, such that the computer, processor other programmable circuitry that execute the code on the machine create the means for implementing various functions, including those described herein.

It is also noted that all or some of the information presented by the example devices and systems discussed herein can be based on data that is received, generated and/or maintained by one or more components of a local or networked system and/or apparatus 200. In some embodiments, one or more external systems (such as a remote cloud computing and/or data storage system) may also be leveraged to provide at least some of the functionality discussed herein.

As described above and as will be appreciated based on this disclosure, embodiments of the present invention may be configured as methods, personal computers, servers, mobile devices, backend network devices, and the like. Accordingly, embodiments may comprise various means including entirely of hardware or any combination of software and hardware. Furthermore, embodiments may take the form of a computer program product on at least one non-transitory computer-readable storage medium having computer-readable program instructions (e.g., computer software) embodied in the storage medium. Any suitable computer-readable storage medium may be utilized including non-transitory hard disks, CD-ROMs, flash memory, optical storage devices, or magnetic storage devices.

### Example Operations

The following refers to the GRACOB device and method, however, one or more devices may be used to perform the operations described such that the operations may be performed by a GRACOB system. Thus, GRACOB device and GRACOB system are used interchangeably. FIGS. 6a and 6b illustrate a series of operations for determining co-fit genes using the GRACOB device. The operations illustrated in FIGS. 6a, 6b may, for example, be performed by, with the assistance of, and/or under the control of a GRACOB device, as described above. In this regard, performance of the operations may invoke one or more of processor 210, memory 220, input/output circuitry 240, communications circuitry 230, GRACOB circuitry 400 (e.g., context determination module 414, analytical engine 416, and/or communications interface 418), and/or GRACOB database 300.

As shown in operation 502 of FIG. 6a, the apparatus 200 includes means, such as processor 210, memory 220, input/output circuitry 240, communications circuitry 230, GRACOB circuitry 400 (e.g., context determination module 414, analytical engine 416, and/or communications interface 418), or the like, for transforming phenotype data using a cumulative distribution function. As shown in operation 504, the apparatus 200 includes means, such as processor 210, memory 220, input/output circuitry 240, communications circuitry 230, GRACOB circuitry 400 (e.g., context determination module 414, analytical engine 416, and/or communications interface 418), or the like, for sorting phenotype data. As shown in operation 506, the apparatus 200 includes means, such as processor 210, memory 220, input/output circuitry 240, communications circuitry 230, GRACOB circuitry 400 (e.g., context determination module 414, analytical engine 416, and/or communications interface 418), or the like, for creating nodes for each consecutive row subset. As shown in operation 508, the apparatus 200 includes means, such as processor 210, memory 220, input/output circuitry 240, communications circuitry 230, GRACOB circuitry 400 (e.g., context determination module 414, analytical engine 416, and/or communications interface 418), or the like, for creating edges between pairs of nodes. As shown in operation 510, the apparatus 200 includes means, such as processor 210, memory 220, input/output circuitry 240, communications circuitry 230, GRACOB circuitry 400 (e.g., context determination module 414, analytical engine 416, and/or communications interface 418), or the like, for removing nodes with a number of consecutive rows under a column threshold. As shown in operation 512, the apparatus 200 includes means, such as processor 210, memory 220, input/output circuitry 240, communications circuitry 230, GRACOB circuitry 400 (e.g., context determination module 414, analytical engine 416, and/or communications interface 418), or the like, for creating one or more maximal cliques in pairs of nodes. As shown in operation 514, the apparatus 200 includes means, such as processor 210, memory 220, input/output circuitry 240, communications circuitry 230, GRACOB circuitry 400 (e.g., context determination module 414, analytical engine 416, and/or communications interface 418), or the like, for extracting biclusters 514.

The GRACOB device and method includes a deterministic graph-based method designed to find maximal constant-column biclusters in any given data matrix. A maximal bicluster means that it is not possible to extend the bicluster by either rows or columns while keeping the same level of specified similarity. Although most interesting variants of the biclustering problems are NP-Complete, the GRACOB device and method takes advantage of the sparsity of biclusters. That is, compared to the size of the input data matrix, the number of biclusters in the matrix is small. In some embodiments, each row represents a gene-deletion strain and each column represents a stress condition.

FIGS. 6a-6b illustrate exemplary operations of the GRACOB device. In FIG. 6a, the data in each column is transformed using a cumulative distribution function, independently, in operation 502. In operation 504, data values in each column are sorted independently from other columns while keeping track of the original row indexes. In operation 506, nodes are created for each consecutive row subset such that the range of their values is at most δ (defined value for how ‘constant’ each column of desired biclusters should be). In this embodiment, a row subset can overlap with other row subsets but cannot be contained by others. In operation 508 of FIG. 6b, an edge is created between any pair of nodes if the nodes are from different columns and share at least r (defined threshold for the smallest number of strains in desired biclusters) rows (i.e. strains). In operation 510, nodes with degree less than c (defined threshold for the smallest number of conditions in desired biclusters) are deleted from the graph. In operation 512, each node is used to grow a clique with its connected nodes (orange circles) while thresholds, r and c, are repeatedly checked to detect future failures as early as possible. In operation 514, row and column index information from each clique is used to extract biclusters from the original data matrix.

In some embodiments, how “constant” the biclusters are to be column-wise in the preprocessed data (see e.g., operation 502) may be determined. The GRACOB device looks at the subsets of strains that maximally satisfy this “constant” requirement inside each independently sorted column. Each of such subsets is defined to be a block, which is a multi-row one-column vector in the corresponding sorted column. Consequently, any column in any potential bicluster is contained by at least one of these blocks (see e.g., operations 504 and 506). The GRACOB device then builds a multipartite graph in which each node is a block and an edge is created between two blocks from two different conditions if the nodes share a sufficient number of strains (see e.g., operation 508). The sufficient number of strains is defined to be the minimum number of strains in a desired bicluster. For instance, if the sufficient number of strains is set to be 1, then every single strain constitutes a constant-column bicluster by definition. If there is a bicluster of n stress conditions, there must exist in the graph a clique of m (m≥n) nodes that contain these n blocks (see e.g., operation 510). The GRACOB device may then determine maximal cliques in this multipartite graph. The GRACOB device divides the problem into smaller ones, and makes use of the characteristics of the data and the requirements of biclusters to search for solutions in a reasonable amount of time (see e.g., operation 512). Biclusters may then be identified inside the maximal cliques (see e.g., operation 514).

The GRACOB device may use three main phases of operations: (i) a pre-processing phase, (ii) a graph creation phase, and (iii) a maximal clique finding phase.

For instance, let G be a set of n mutant strains, each of which is a single gene knock-out mutation, and C be a set of m environmental stress conditions. The elements of the growth phenotype data matrix A(_{n×m}) may be referred to as aᵢⱼ, where aᵢⱼ is a real value that represents the growth of the ith mutant under the jth stress condition where i≤n and j≤m.

To define a constant-column bicluster, the three parameters may be determined. The first parameter is the range threshold, δ, to define how “constant” each column is in the desired biclusters. For example, if δ is set to be 0, biclusters within which each column contains data with exactly the same value will be found. The second one is the row threshold, r, to define the minimum number of strains (or genes) that each bicluster must have. If r is set to be 1, each row becomes a trivial constant-column bicluster because each column for the same row has 0 variance. The third parameter is the column threshold, c, to define the minimum number of conditions each desired bicluster must contain. If c is set to be 1, the biclusters will be a part of a single column.

Once the requirements are provided, let I⊆G and J⊆C. I is a set of co-fit genes across the J conditions if the mutant strains had a similar growth phenotype across these conditions such that:

ƒ(aᵢ₂ⱼ)−δ≤ƒ(aᵢ₁ⱼ)≤ƒ(aᵢ₂ⱼ)+δ (1a)

|ƒ(aᵢ₂ⱼ)−ƒ(aᵢ₁ⱼ)|≤δ (1b)

I and J specify a desired constant-column bicluster if the following conditions are satisfied:

|ƒ(aᵢ₁ⱼ)−ƒ(aᵢ₂ⱼ)|≤δ, (2)

|I|≥r, (3)

|J|≥c, (4)

where i₁, i₂∈I and j∈J, δ is a similarity tolerance threshold. The “|x|” denotes the cardinality of a set and “ƒ(x)” is a transformation function as discussed herein. In particular, “ƒ(x)” transforms the relative growth data to another space where differences between original values can be measured using Euclidean distance function. The submatrix (I, J) is a bicluster. Eq. (2) ensures that the values within each column of the bicluster are similar, whereas Eq. (3) and Eq. (4) ensure only non-trivial biclusters are reported. The GRACOB device thereby finds all I and J that satisfy these conditions, and there is no I′ and J′ such that I⊆I′ and J⊆J′ that satisfies these conditions, e.g., only maximal constant-column biclusters are returned.

The GRACOB device may then transform the data in each stress condition based on a cumulative distribution and may then create blocks (or “nodes”). The input growth phenotype data may be assumed to follow a standard normal distribution where the data has been z-score normalized inside each column. As most of the outlier data points are distributed along a long range of values, the outlier data points are considered to show similar phenotypes, e.g., growth is extremely sensitive (negative outliers) or stable (positive outliers) with respect to environment conditions. Thus, there is a need to transform the data into another space which preserves the similarity of these values. A cumulative distribution function “CDF” may be applied to each column, independently, in the input matrix to transform the data. Consequently, data points in the tail of each side may be assigned very close values. The right panel of FIG. 6a illustrates the distribution of the values for a column after the CDF transformation.

The GRACOB device may then create blocks that are the nodes for the multipartite graph. The data is sorted (see e.g., operation 504) and then each column is linearly scanned to provide all of the blocks within the range of values at most δ. These blocks are used as the (unit) nodes for the following operations (see e.g., operation 506).

For instance, in some embodiments, let A(_{n×m}) be a matrix of growth phenotype data with n Δ-genes and m environmental stress conditions. For all i≤n and j≤m, the following transformation matrix is obtained: A′=cdƒ(A) such that a′ᵢⱼ=cdƒ(aᵢⱼ, μ, σ) and

$$cdf ( {a}_{ij} , μ , σ ) = \frac{1}{σ \sqrt{2 π}} {\int }_{- ∞}^{{o}_{ij}} \text{?} dx \\ \text{?} \text{indicates text missing or illegible when filed} \qquad ( 5 )$$

In some embodiments, after the transformation of values, the top and bottom 16% of the values in each column are kept in order to better detect conditionally essential and dispensable co-fit genes. The top and bottom 16% of the values in each column after the CDF transformation correspond to the values beyond one standard deviation from the mean in the original column, which has a normal distribution. In some embodiments, the GRACOB device and method does not use this filtering. In some embodiments, the filtering is used as the inclusion of those genes with moderate loss-of-function effects could lead to an increase in the number of noisy biclusters with unrelated gene functions. This is because such moderate effects could be explained by a number of causes such as experimental noise and cross talk. Thus, while such a treatment will increase the number of biclusters found, the inclusion of those genes would unlikely contribute to a better characterization of the function of genes.

Blocks may then be created for the multipartite graph. In some embodiments, a user may provide the range threshold on CDF transformed values. In some embodiments, the range may be about 0.01 to about 0.10, such as about 0.05. In some embodiments, the row threshold, r, may be provided. A meaningful value of r may depend on the size of the data matrix, the user's interest, and δ. In some embodiments, the value of r is set to ensure the statistical significance of the discovered biclusters. Based on the data matrix of size N×M, for a given value of r, the probability that a bicluster of size r appears in a random data matrix of size N×M may be determined. The probability can be predetermined and used to pick the value of r such that such probability satisfies some significance threshold, e.g., <0.001. In some embodiments, r may be set to be 2, 3, 4, 5, 6, 7, 8, 9, 10, etc. For instance, r may be set to be 4.

For every column, all values may be sorted in an ascending order and the associated original row indexes may be obtained (see e.g., operation 504 of FIG. 6a). Each sorted column may then be scanned for consecutive blocks of rows that satisfy the following requirements: 1) a block contains at least r strains; 2) the largest difference among the values in each block is at most δ; and 3) a block can overlap with other blocks, but cannot be contained by any other block. Scanning can be done in linear time with respect to the size of the columns. These blocks are used as the (unit) nodes for the following phases (see e.g., operation 506 of FIG. 6a).

The GRACOB device may then create edges between the blocks (unit nodes). The edges are not weighted but rather labeled by the shared subsets of strains. There is no edge created between nodes from the same condition, and the cardinality of the shared subset of an edge must be at least r. The complexity of such a process is O(S²) where S is the total number of nodes. With genome-wide growth phenotype data, S can be in the order of millions and O(S²) runtime becomes infeasible. However, the GRACOB device may be designed to use a divide-and-conquer approach by repeatedly using the defined thresholds c and r to reduce the search space, and thus reduce the practical runtime. All of the blocks inside each column may be merged into a super-node and edges may be created among these super-nodes. The GRACOB device then divides the super-nodes into non-overlapping child nodes, each of which is a subset of blocks and inherits the edges from its parent node, unless the cardinality (i.e. number of genes) of the edge is below r, which means this edge will never be a part of a meaningful bicluster. If such a non-overlapping split is not feasible, then the GRACOB device splits in the middle. Meanwhile, the GRACOB device deletes all the nodes that have a degree below c, which means the blocks in those nodes will never be a part of bicluster with at least c stress conditions. The GRACOB device recursively performs the splitting until each node is a block.

In some embodiments, the column threshold, c, may be determined. Similar to r, a meaningful value of c may depend on the size of the data matrix, the user's interest, and r. In some embodiments, the treatment of c can be similar to that of r. In some embodiments, r and c may be set independently. When the data matrix size is fixed, for each pair of r, c values, the probability of seeing a constant-column bicluster of at least r rows and c columns in a random matrix of the same size can be determined. In some embodiments, only the settings that satisfy certain statistical significance may be accepted. In some embodiments, c can be set to be four. In some embodiments, values for r and c may be suggested to a user.

For each column, a super-node that is labeled with the union of all the blocks may be created. A tree rooted at this super-node may be built for each column. In some embodiments, the number of super-nodes may be small, thus a pairwise check between the super-nodes may be conducted and an edge between any pair if and only if the cardinality of the shared strains between the two super-nodes is at least r may be prepared.

Starting with the super-node (as the root), a tree may be constructed through recursive splitting. For each node, if the node contains more than one unit node, a non-overlapping splitting point to split this node into two child nodes may be determined. Each child node may contain a subset of the unit nodes from its parent and the child node may inherit its parent's edges. If no non-overlapping split exists, the node may be split in the middle. Since the unit nodes in the parent set may already be ordered from the previous phase, this splitting can be done in linear time. This can help reduce overlaps between nodes, and may consequently reduce the number of required checks and created edges in the following iterations.

For every edge of the current child node, the node, p, at the other end of this edge may be checked. If p is a parent node, an edge may be created between this child node and the child nodes of p if they share at least r strains. If p does not have child nodes, the edge between this child node and p may be kept if they share at least r strains (see e.g., operation 508 in FIG. 6b).

After updating the edges of all the nodes, all nodes that are connected to fewer than c−1 other nodes may be eliminated as such nodes may not be part of a bicluster of at least c conditions (see e.g., operation 510 of FIG. 6b). The above may be repeated until no splitting can be done or all the nodes are eliminated, which means all the remaining nodes are unit nodes.

The GRACOB device may find and return all maximal cliques, from which biclusters can be extracted. Existing general-purpose maximal clique finding methods do not suffice for determining co-fit genes. In contrast, the GRACOB device and method starts from each remaining unit node from the previous phase, and sequentially grows cliques seeded from this node by gradually adding connected nodes to the existing cliques. The minimum row and column thresholds, r and c, may be used to detect future failures as early as possible and to eliminate those cliques that have no hope to grow to the required size.

In some embodiments, for each remaining unit node from the previous phase, a subgraph may be created that consists of the node as the seed node. Each subgraph may contain the following information: 1) the set of strains in this subgraph, e.g., at the beginning, it only contains strains within the seed node, 2) the maximum column index of all the nodes in this subgraph, which is initialized to be the column index of the seed node, and 3) the successor set, which is initialized to contain all the nodes connected to the seed node.

For every subgraph, all successors may be iterated through. If the index of the column of the successor is larger than the maximum index of this subgraph, then the cardinality of the intersection between the strain set of the subgraph and that of the successor is check to be at least r, and if the cardinality of the intersection between the successor set of the subgraph and that of this successor is at least c-|s|-1, where |s| is the cardinality of this subgraph. If both are satisfied, a new subgraph is created which contains this subgraph and this successor, and the information for this new subgraph is updated accordingly. If the subgraph cannot grow after checking all the successors, the size of this subgraph is check to be at least c. If so, the subgraph is added to the result set. If not, the subgraph may be deleted.

The above may be repeated until no more subgraphs can be grown. All the remaining subgraphs are thus maximal cliques of the multipartite graph (see e.g., operation 512 of FIG. 6b). All biclusters with at least r rows and at least c columns may be enumerated inside the cliques and returned (see e.g., operation 514 of FIG. 6b).

In some embodiments, the GRACOB device and method may determine all maximal biclusters in the given growth phenotype dataset, under the given thresholds, δ, r, and c. Neither the divide-and-conquer method used in the graph creation phase nor the early detection of failures operation used in the maximal clique finding phase negatively affects the optimality of the search.

Without intending to be limited by theory, the GRACOB device and method may provide the first device and method specifically designed for mining co-fit genes from growth phenotype profiling data. The GRACOB device and method may discover all the maximal constant-column biclusters, fully taking advantage of the properties of such data. The identified co-fit genes may guide the systems biology and synthetic biology studies and industries by narrowing down to important candidates on the growth of the microorganisms.

### EXPERIMENTAL

Following Prelić et al. (2006); Li et al. (2009); Eren et al. (2013), the GRACOB device and method was validated using a variety of synthetic datasets, where different types of implanted biclusters, different levels of noise, and different degrees of bicluster overlaps were simulated.

Each of the simulated scenarios were specified by three GRACOB parameters to determine whether the implanted biclusters are constant biclusters or constant-column ones, whether the data matrix is permutation-free or permutation-specific, and whether the noise level is gradually changed or the overlapping degree is gradually changed. Consequently, all the eight combinations of these three GRACOB parameters were simulated. For each setting of the noise level or overlapping degree, 10 random simulations were conducted. All the results reported later are the average performance over the 10 simulations for each setting.

The eight scenarios include: 1) Ten constant biclusters (five with value 2 and five with value −2) were implanted in a data matrix of size 100×50. There is no permutation done on the data matrix. The variance for the noise was changed from 0 to 0.25, with step size 0.05. FIG. 9 part (1a) illustrates a typical case for this scenario. 2) Ten constant-column biclusters were implanted in a data matrix of size 100×50. The odd columns of the implanted biclusters were set to the values of −2−i/50 where i is the column index. The even columns of the biclusters were set to the values of 2+i/50. The same noise change was applied as in scenario 1 (see FIG. 9 part (2a)). 3) Ten constant biclusters with value −2 were implanted in a matrix of size 100×100. Here a larger matrix was used to allow implanted biclusters to have sufficiently large overlaps in rows and columns. The variance of the background noise was set to 0.02. The bicluster sizes were gradually enlarged to make the number of overlapping rows and columns between two consecutive biclusters change from 0 to 8, with step size 1 (see FIG. 9 part (3a)). 4) Ten constant-column biclusters were implanted in a data matrix of size 100×100, with odd columns having values −2−i/100 and even ones with 2+i/100. The background noise and overlapping degree were set to be the same as in scenario 3 (see FIG. 9 part (4a)). 5-8) These four scenarios were the random permutations of the data matrices generated by scenarios 1-4, respectively, which are illustrated in FIG. 9 parts (5a)-(8a). In addition to the noise added to the implanted biclusters, the background noise was set to the non-biclusters regions to be white Gaussian noise, with mean zero and variance one.

FIG. 9 illustrates the performance comparison on the synthetic data sets. FIG. 9 parts (1a)-(8a) illustrate the typical data sets for the 8 scenarios. FIG. 9 part (1a) illustrates constant biclusters with changing noise level; part (2a) illustrates constant-column biclusters with changing noise level; part (3a) illustrates constant biclusters with changing overlapping degree; part (4a) illustrates constant-column biclusters with changing overlapping degree; part (5a) illustrates constant biclusters with changing noise level with random permutation; part (6a) illustrates constant-column biclusters with changing noise level with random permutation; part (7a) illustrates constant biclusters with changing overlapping degree with random permutation; and part (8a) illustrates constant-column biclusters with changing overlapping degree with random permutation. FIG. 9 parts (1b)-(8b), (1c)-(8c), and (1d)-(8d) illustrate the precision, recall, and F1-score (averaged over 10 simulations for every setting of noise level and overlapping degree) of different methods for the eight scenarios, respectively. For visualization purpose, only the values above 0.5 are shown. FIG. 9 part (9a) illustrates a sensitivity analysis of the GRACOB device and method with respect to the parameters r and c on the part (8a) scenario. FIG. 9 part (9b) illustrates the F1-score of different methods on the part (8a) scenario with respect to the different data matrix size. FIG. 9 part (9c) illustrates the runtime of different methods on the (8a) scenario with respect to the different data matrix size.

Since the ground-truth biclusters are known for the synthetic datasets, recall, precision, and F1-score were used to measure the performance Given the set of predicted biclusters by a method, the best matching between this set and the set of the ground-truth biclusters was first found. A Munkres assignment type of approach was applied. Let B* denote the set of the ground-truth biclusters and b*∈B* denote any ground-truth bicluster. Let B denote the set of the predicted biclusters and b∈B denote any predicted bicluster. A bipartite graph was built between B* and B, where each node was a bicluster, and each edge between b* and b was defined to be the shared area between the two biclusters over the area of b*. The maximum weighted bipartite matching problem was then solved to find the best matching between the two sets of biclusters. Then, for each corresponding pair of the true and the predicted biclusters, b* and b, define TP to be their overlapping area. Then recall was defined as TP/|b*| where |b*| is the area of b*, and precision was defined as TP/|b|. Since neither recall nor precision alone can comprehensively reveal a method's overall performance, the F1-score was calculated, which is defined as the harmonic mean of the recall and precision.

The results (see FIG. 9) show that among the 14 compared methods, ISA, QUBIC, and the GRACOB device and method were all able to detect both constant biclusters and constant-column biclusters well and can tolerate noise. However, when the overlapping degree of the implanted biclusters was high, the GRACOB device and method was the only one that can almost perfectly identify all the implanted biclusters.

As shown in FIG. 9, among the 14 methods, only four methods, ISA, QUBIC, SAMBA and the GRACOB method, were able to achieve good performance (at least 0.5 in recall, precision, or F1-score) for permutation-free data sets (FIG. 9(1a)-(4a)). This is consistent with the reported performance of different methods in previous comparative studies. ISA, QUBIC and the GRACOB device and method can perfectly predict all the implanted non-overlapping biclusters regardless of the noise level (FIG. 9(1a-d) and (2a-d)), whereas the performance of SAMBA was reasonable but inferior to them. However, the performance of ISA, QUBIC, and SAMBA dropped substantially when overlapping degree increases (FIG. 9 (3a-d) and (4a-d)), while the GRACOB device and method managed to maintain nearly perfect performance.

For the same synthetic data sets but with random permutations of rows and columns (FIG. 9 (5a)-(8a)), SAMBA no longer performed well which suggests its deficiency in recovering biclusters from randomly organized data sets. In contrast, ISA, QUBIC and the GRACOB device and method were insensitive to the permutation. CPB had quite unstable performance under different scenarios. Although it outperforms ISA and QUBIC in some situations with high overlapping degrees, its performance was not comparable with that of the GRACOB device and method. In summary, when overlapping degree is not high, ISA, QUBIC and the GRACOB device and method were all able to reliably detect both constant biclusters and constant-column biclusters. When overlapping degree is high, the GRACOB device and method was the best option.

Sensitivity analysis on the GRACOB device and method was performed with respect to the parameters r (minimum number of rows for biclusters) and c (minimum number of columns), and the GRACOB device and method showed strong robustness to these parameters (FIG. 9 (9a)). The three best performing methods were then further evaluated with respect to the increasing size of the input data matrix. In terms of F1-score, the GRACOB device and method was very stable whereas ISA and QUBIC were less (FIG. 9(9b)). In terms of the runtime, the GRACOB device and method had a similar runtime to QUBIC, while both were faster than ISA (FIG. 9 (9c)).

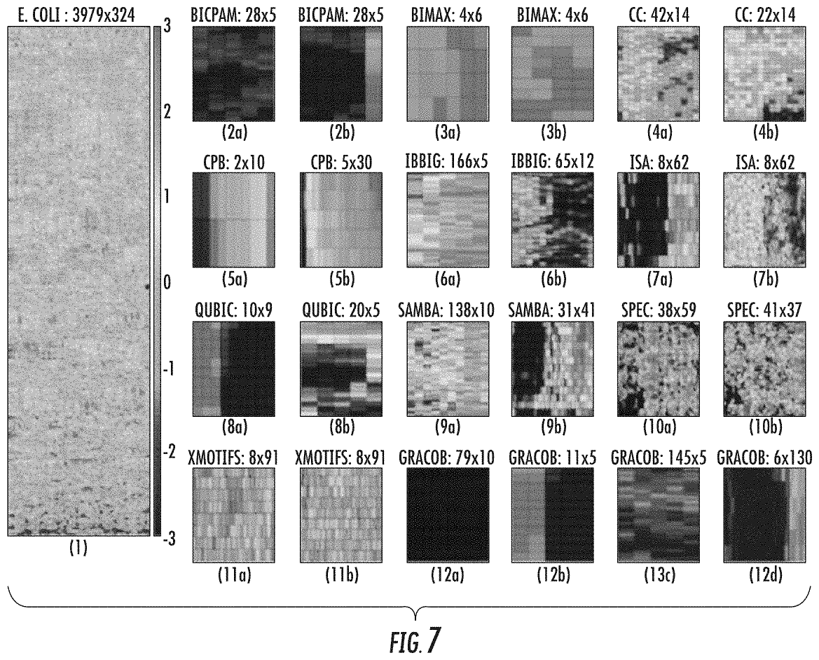

To comprehensively evaluate the performance of the GRACOB device and method, three recently measured growth/fitness phenotype datasets were used. The first growth/fitness phenotype dataset was the genome-wide growth phenotype dataset of E. coli (Nichols et al., 2011). This dataset consists of fitness data for 3979 mutant strains, each of which was measured under 324 different stress conditions. Each fitness value in the data matrix represented the relative growth rate of a given gene-knockout strain under a given stress condition, which was normalized column-wise to follow the unit normal distribution (Nichols et al., 2011). FIG. 7 part 1 shows this growth phenotype dataset.

In particular, FIG. 7 provides a heatmap visualization of the E. coli growth phenotype data and the representative biclusters detected by the 11 methods. FIG. 7, part (1) is the heatmap visulalization for the capped data matrix for the E. coli growth phenotype dataset with 3979 strains and 324 stress conditions. All of the values larger than 3.0 were capped as 3.0 and all of the values smaller than −3.0 were capped as −3.0, for visualization purposes. FIG. 7, parts (2)-(12) are the representative biclusters detected by BicPAM, Bimax, CC, CPB, iBBiG, ISA, QUBIC, SAMBA, Spectral, xMOTIFs and GRACOB, respectively. For each method, the predicted biclusters that have consistent patterns which appear many times in the results of the method were selected. For visualization purposes, rows and columns of each bicluster were organized by hierarchical clustering (Eisen et al., 1998). That is, genes with similar values were clustered on the Y-axis and conditions with similar values were clustered on the X-axis.

The second growth/fitness phenotype dataset was the DNA tag-based pooled fitness assay dataset for Shewanella oneidensis MR-1, a Gram-negative γ-proteobacterium (Deutschbauer et al., 2011). The dataset contained the mutant fitness for 3355 nonessential genes under the 195 pool fitness experiments.

The third growth/fitness phenotype dataset was the growth response dataset for Saccharomyces cerevisiae (Hillenmeyer et al., 2008). The dataset contained 5337 heterozygous gene deletion strains over 726 conditions.

The real growth phenotype data did not have known ground-truth biclusters. Thus, to measure the performance of biclustering methods on the real data, four performance measures were defined. Since each biclustering method can discover a large number of biclusters in a given dataset, the measures considered the performance based on multiple biclusters. If the number of predicted biclusters was smaller than 100, all were kept. Otherwise, the top 100 largest biclusters for evaluation were kept. In order to reduce the bias caused by highly overlapping biclusters in evaluation, the returned biclusters were sorted by size in a descending order. Only the biclusters that share less than 30% of the size of this bicluster with any previously selected bicluster were then kept until 100 biclusters were selected.

The first measure was the average column-wise standard deviation. The mean of the column-wise standard deviation for each bicluster was calculated, and then the average of this value over all the predicted biclusters was calculated. The second measure was the average size of the predicted biclusters, where the size of a bicluster was measured by the number of rows times the number of columns. Thus, a method that simultaneously reports a small average standard deviation and a large average bicluster size was considered to be useful.

Furthermore, each bicluster was subject to two enrichment analyses, using pathway information from the KEGG database (Kanehisa and Goto, 2000) and gene ontology (GO) terms, respectively. For each of the predicted biclusters of a method, the set of genes that correspond to the strains of this bicluster was found, and all the annotated pathways that contained at least one gene from this gene set was searched. Then, the probability, i.e. P-value, of randomly finding these genes for each pathway was calculated with the hypergeometric calculation (Li et al., 2009). As used herein, the precision of a method is the ratio of biclusters which have at least one significant pathways (i.e. P-value smaller than a given threshold, e.g. 10⁻⁷, 10⁻⁶, 10⁻⁵, 10⁻⁴, or 10⁻³) to the total number of selected biclusters for that method. The number of selected biclusters for any method was at most 100 as explained above. The same procedure was done for the GO term enrichment analysis, and the GO-level precision for different methods is reported as the fourth measure.

### Results and Discussion

The GRACOB device and method was compared with the 13 representative biclustering methods introduced in the related work. For each experiment, the input data was transformed and preprocessed following the requirements of the respective method. The parameter settings for the 13 methods were searched and optimized based on the recommended use from the respective papers.

Some representative biclusters predicted by 11 methods on the E. coli dataset are illustrated in FIG. 7 parts (2)-(12). BBC and FLOC failed to detect any bicluster on these large growth phenotype datasets in 3 hours, and Plaid only predicted less than three biclusters and thus was not included in the analysis for the sake of comparison. It is clear that the biclusters detected by Bimax were purely constant, whereas the ones detected by CPB, iBBiG, ISA, and SAMBA tended to have relatively constant columns, although the methods were still far less constant than the ones detected by BicPAM and the GRACOB device and method. Among these four methods (i.e., CPB, iBBiG, ISA, and SAMBA), CPB and iBBiG had relatively lower column-wise standard deviation, whereas ISA and SAMBA tended to detect bigger biclusters. It is worth noting that biclusters predicted by Bimax were not only smaller than those predicted by the GRACOB device and method, but the biclusters also contained only large positive values. This result was due to the required binary discretization step in Bimax. Among the biclusters returned by the GRACOB device and method, about 62% consisted of only conditionally essential genes (i.e. biclusters in the blue color), 20% consisted of only conditionally dispensable genes (i.e. biclusters in the red color), and 18% consisted of genes that are essential under certain conditions but dispensable under some other conditions (i.e. biclusters with mixed colors).

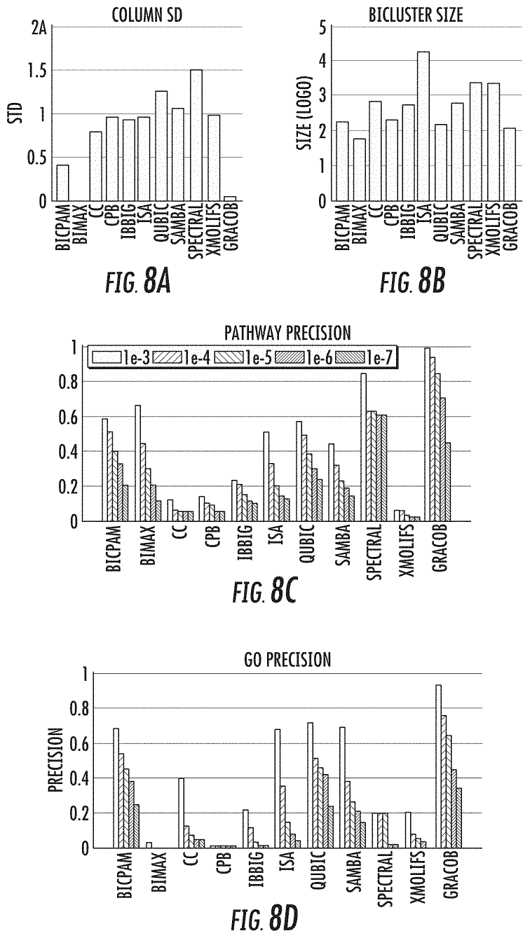

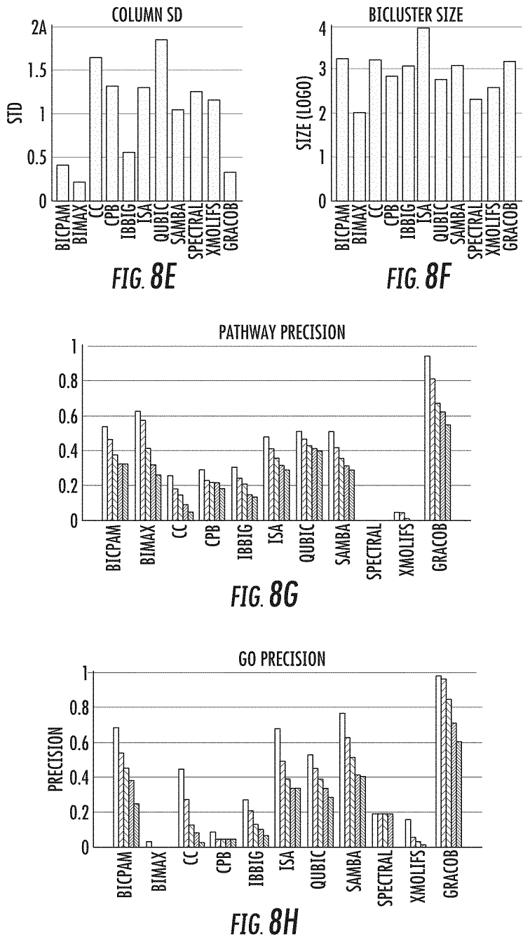

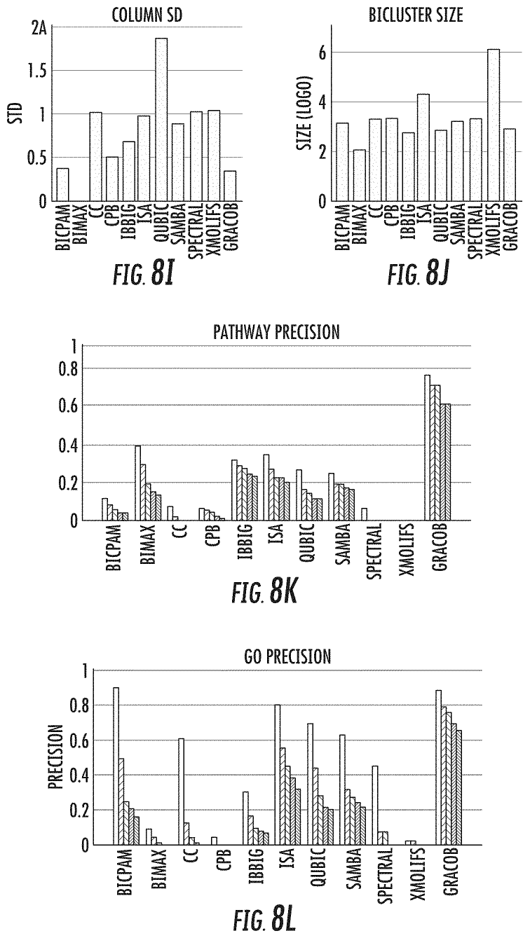

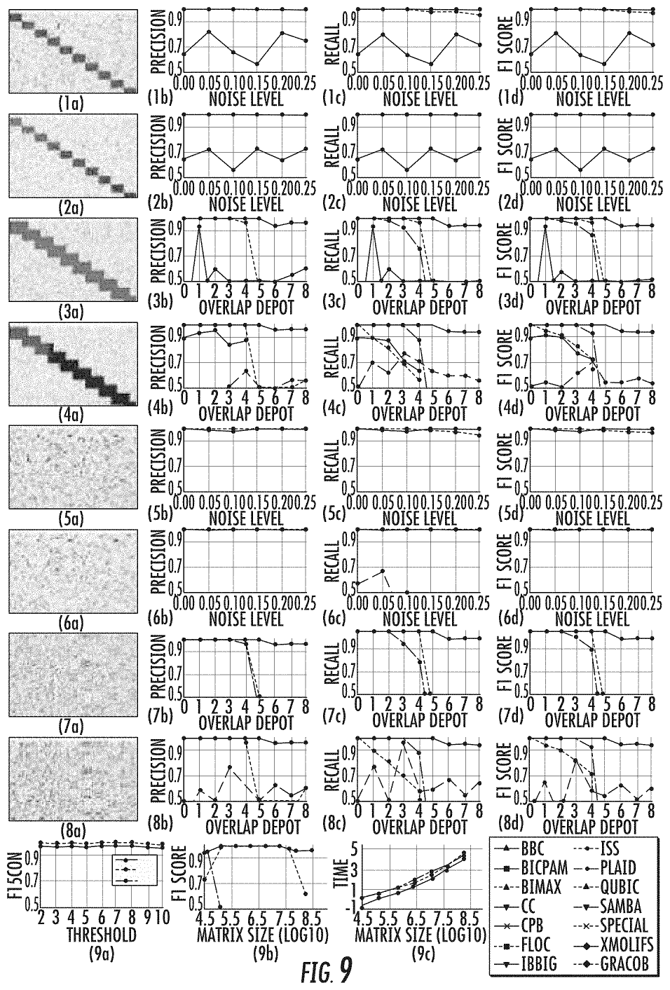

In terms of the average column-wise standard deviation, as expected, Bimax and the GRACOB device and method had the lowest column-wise variance, followed by BicPAM (FIGS. 8a, 8e, and 8i). However, the average bicluster size of the GRACOB device and method was one order of magnitude bigger than that of Bimax (FIGS. 8b, 8f, and 8j). Although ISA, Spectral and xMOTIFs can return large biclusters, the biclusters were very impure. Overall, the GRACOB device and method had a remarkably strong ability to discover maximal constant-column biclusters. As shown in FIGS. 8c, 8g, and 8k, the GRACOB device and method had the highest percentage of significantly enriched KEGG pathways among all the 11 methods, under almost all the different significance levels. The only exception was for the E. coli dataset, when the significance threshold was below 1E-7, the precision of the GRACOB device and method was slightly lower than that of Spectral. The average precision of the GRACOB device and method under the five significance thresholds (10⁻³, 10⁻⁴, 10⁻⁵, 10⁻⁶, and 10⁻⁷) were 0.90, 0.82, 0.75, 0.64 and 0.53, respectively, whereas that of the second best method were 0.56 (Bimax), 0.44 (Bimax), 0.32 (QUBIC), 0.27 (QUBIC) and 0.24 (QUBIC), respectively. These results show that for this analysis the GRACOB device and method was at least 61%, 86%, 134%, 137% and 121% more precise than any other biclustering method in terms of KEGG pathways under the five significance levels, respectively.

FIG. 8a-8l provides a performance comparison of the 11 methods on the E. coli, proteobacteria and yeast growth phenotype datasets. FIGS. 8a, 8e, and 8i illustrate the average column-wise standard deviation on the three datasets, respectively. FIGS. 8b, 8f, and 8j illustrate the average size of the returned biclusters on the three datasets, respectively. FIGS. 8c, 8g, and 8k illustrate the KEGG pathway-level precision under five significance levels on the three datasets, respectively. FIGS. 8d, 8h, and 8l illustrate the GO term-level precision under five significance levels on the three datasets, respectively.

Similar conclusions can be drawn on the GO term-level precision. The GRACOB device and method was more precise than the other methods under almost all the situations (see e.g., FIGS. 8d, 8h, and 8l), except for the yeast data when the significance level was 10′, the GO-level precision of the GRACOB device and method (0.89) was slightly lower than that of BicPAM (0.91). The average precision of the GRACOB device and method over the three datasets under the five significance levels were 0.93, 0.84, 0.76, 0.62 and 0.54, which show that for this analysis the GRACOB device and method was 26%, 71%, 105%, 88% and 108% more precise than the second best method, respectively, which were BicPAM (0.74), BicPAM (0.49), QUBIC (0.37), QUBIC (0.33) and SAMBA (0.26), respectively.

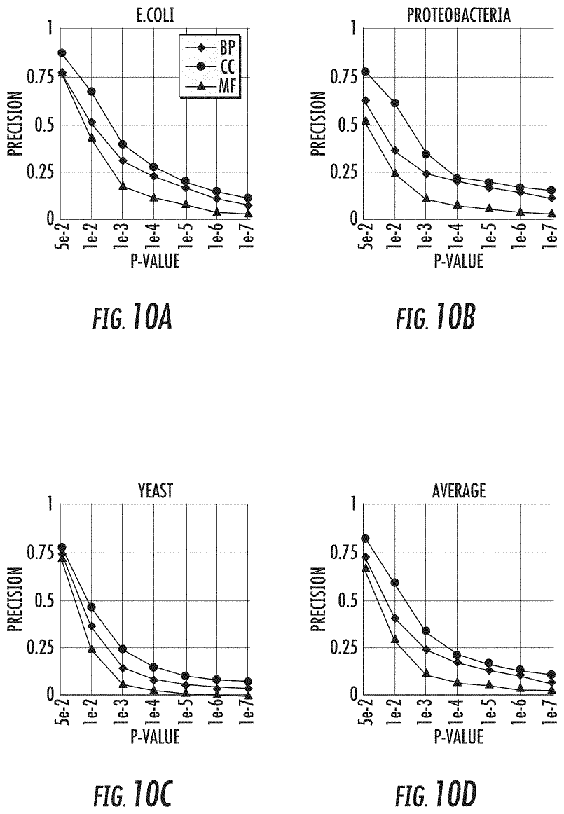

The enrichment over the three branches of GO terms (Biological Process, Cellular Component and Molecular Function) was also analyzed. The results revealed that the highest percentage of enriched GO terms among the co-fit genes detected by GRACOB biclusters belonged to the Cellular Component (CC) branch in all the analyzed species. This is in agreement with the findings in Hillenmeyer et al. (2010) that co-fitness is a powerful tool to predict cellular functions. FIGS. 10a-10d show the GO term enrichment precision under different significance levels for the three branches of the GO hierarchy for E. coli, proteobacteria, and yeast, respectively. In particular, the figures illustrate the GO term enrichment precision per GO category as predicted by the GRACOB device and method for E. coli (FIG. 10a), proteobacteria (FIG. 10b), yeast (FIG. 10c), and average over all the data sets (FIG. 10d). The circle, triangle, and diamond lines represent GO terms under Cellular Component (CC), Molecular Function (MF), and Biological Process (BP), respectively. The precision was defined by TP/P, where TP is the number of GO terms for the specific GO branch that are enriched at the given significant level in any of the top 100 biclusters detected by the GRACOB device and method, and P is the number of GO terms for the specific GO branch that are annotated by any gene of the top 100 biclusters detected by the GRACOB device and method.

Parameter sensitivity analysis of the GRACOB device and method over the E. coli dataset was also conducted. GRACOB was very stable with respect to the changes of parameters r and δ, while less so when c increased.

The parameter sensitivity analysis was performed of the GRACOB device and method with respect to the three parameters, r, c, and δ, where r is the minimum number of rows for the detected biclusters, c is the minimum number of columns for the detected biclusters, and δ is the range of the values inside each column of the detected biclusters after the values are converted by CDF transformation.

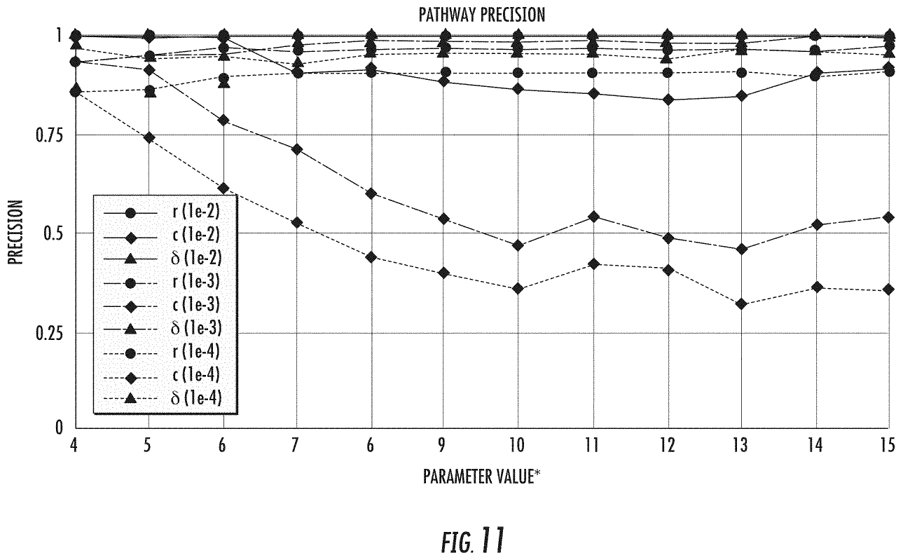

FIG. 11 illustrates the parameter sensitivity analysis for the GRACOB device and method in terms of the KEGG pathway-level precision of the detected biclusters on the E. coli data set. Circle, diamond, and triangle curves represent the precision of the GRACOB device and method when the parameter, r, c, and δ, is changed, respectively. Solid, dash-dot, and dotted curves represent the precision of the GRACOB device and method under different significance level thresholds, 1e-2, 1e-3, and 1e-4, respectively. The values on the x-axis are the values for r, c, and 100δ.

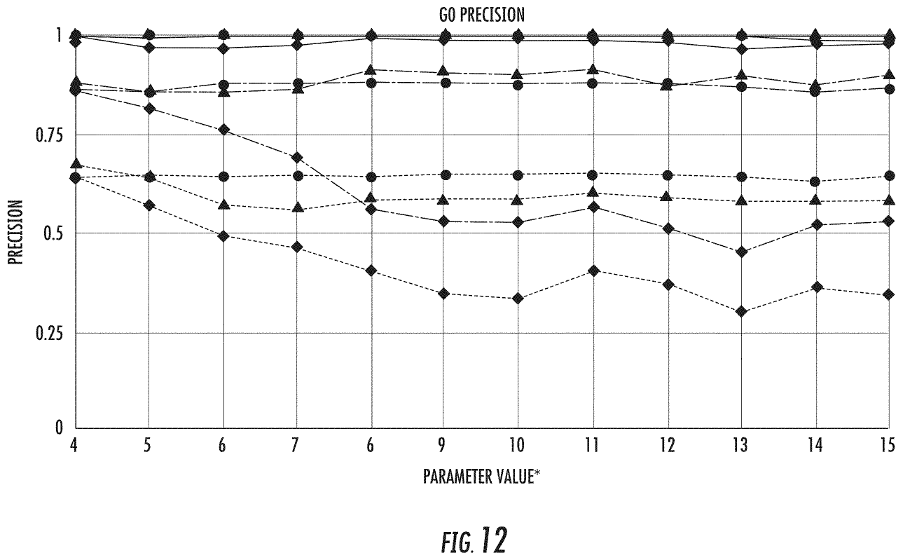

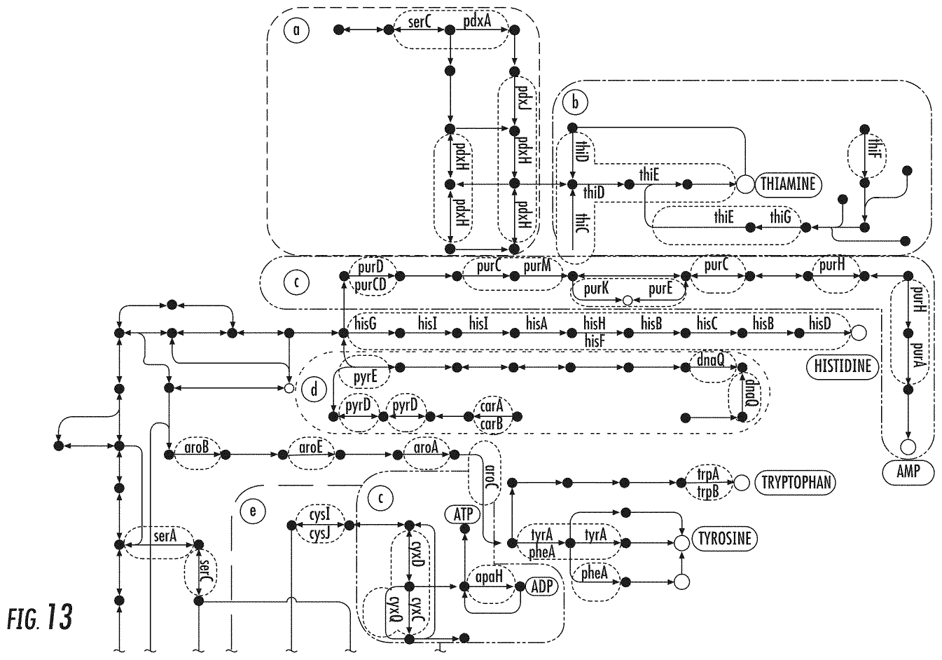

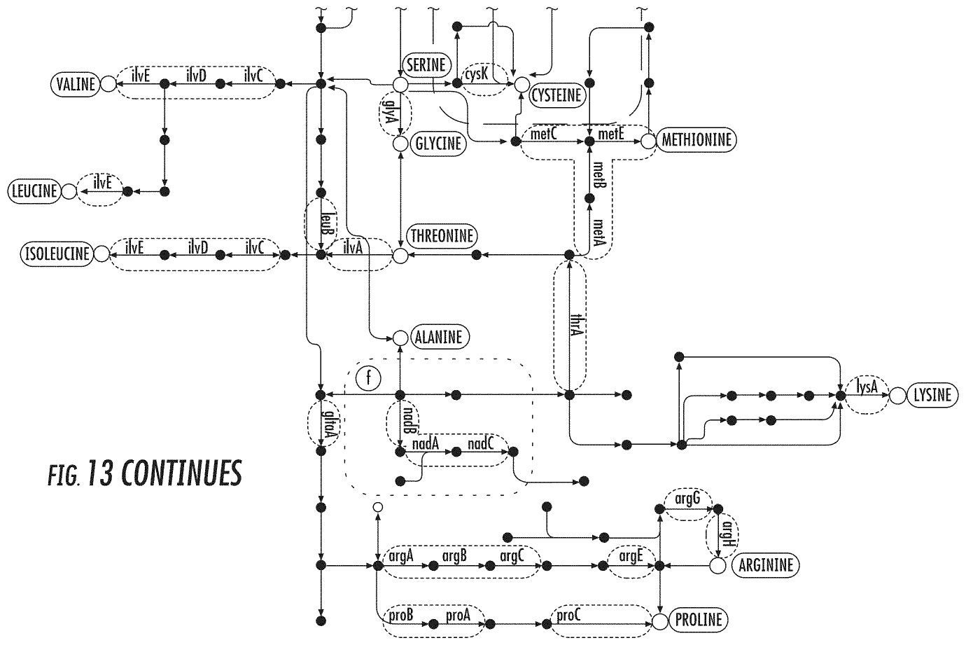

FIG. 12 illustrates the parameter sensitivity analysis for the GRACOB device and method in terms of the GO term-level precision of the detected biclusters on the E. coli data set. Circle, diamond, and triangle curves represent the precision of the GRACOB device and method when the parameter, r, c, and δ, is changed, respectively. Solid, dash-dot, and dotted curves represent the precision of the GRACOB device and method under different significance level thresholds, 1e-2, 1e-3, and 1e-4, respectively. The values on the x-axis are the values for r, c, and 100δ.

As shown in FIG. 11 and FIG. 12, the performance of the GRACOB device and method may be quite stable when r and δ are changing. Such stability makes sense because the number of genes in a group of co-fit genes is often bigger than 10 to be able to function together for conditional essentiality or conditional dispensability, which means the GRACOB device and method may not be sensitive to r in some embodiments. Since the range δ may be applied after the CDF transformation, and the GRACOB device and method may focus on the top and bottom 16% of the values (e.g., the values beyond one standard deviation from the mean in the original column), the GRACOB device and method may not be sensitive to δ in some embodiments either. However, when c increases, the precision of the GRACOB device and method may have a clear decrease, especially for the more stringent significance level.

The largest bicluster that the GRACOB device and method detected in the E. coli growth phenotype dataset is shown in FIG. 7 part 12a. The bicluster grouped 79 gene knock-out strains under 10 stress conditions (see Tables S1 and S2 below for details). The knock-out of any of these 79 genes lead to significantly reduced cell growth under these 10 conditions, although none of them is an essential gene. The 10 conditions consisted of seven carbon-source conditions, one nitrogen-source condition, and two ferrous sulfate-source conditions. These sources may be transported and metabolized by pathways that require amino acids, purines, pyrimidines and cofactors to be synthesized. Thus, deletions of genes involved in such pathways may be expected to impact the cell growth under these conditions.

TABLE S1
Mutant genes from bicluster #1
Δ Genes

apaH
argA
argB
argC
argE
argG
argH
aroA
aroB
aroC
aroE
carA
carB
cysB
cysC
cysD
cysG
cysI
cysJ
cysK
cysQ
dnaQ
gcvR
gltA
glyA
hisA
hisB
hisC
hisD
hisF
hisG
hisH
hisI
ilvA
ilvC
ilvD
ilvE
leuB
lysA
lysR
metA
metB
metC
metE
metF
metR
nadA
nadB
nadC
pdxA
pdxH
pdxJ
pheA
proA
proB
proC
purA
purC
purD
purE
purH
purK
purL
purM
pyrC
pyrD
pyrE
serA
serC
thiC
thiD
thiE
thiF
thiG
thrA
trpA
trpB
tyrA
ycdY

TABLE S2
Bicluster #1 stress test conditions

| Stress test label | Test family |
|---|---|
| Acetate | C-Source |
| N-Acetylglucosamine | C-Source |
| Glucosamine | C-Source |
| Glucose | C-Source |
| Glycerol | C-Source |
| Maltose | C-Source |
| Succinate | C-Source |
| Nh4cl | N-Source |
| High-Fe | Metal |
| Low-Fe | Metal |

Among the 79 genes found in one embodiment, there were 74 enzyme coding genes, of which 72 were closely connected through KEGG pathways as can be seen in FIG. 13 (see Case Study No. 1 below). In fact, 70 genes (88.6% of the genes in this bicluster) are involved in metabolic pathways. This is statistically significant because only 15.2% of the total 3979 genes are known to be involved in metabolic pathways (see Tables S3 and Case Study No. 1 for details). The second and third most significant KEGG pathways in this bicluster were biosynthesis of secondary metabolites and biosynthesis of amino acids, in which 44 and 41 of the genes are involved, respectively. This is interesting because secondary metabolites generally do not play a role in growth under the normal condition. However, it is discovered that the metabolites can be important in survival of organisms because the metabolites are involved in physiological functions like stress-response.

FIG. 13 illustrates a pathway map of genes from the case study bicluster as shown in FIG. 7 part (11a). Highlighted by ovals are the reactions catalyzed by enzymes coded by genes from the bicluster, in which their labels are attached to the edges representing the reactions. The small circles are intermediate products of reactions and the large circles are selected main products of pathways. The labels of these products are given and underlined. None labeled edges are reactions found in the used pathway maps from KEGG. Most of the map elements were obtained from KEGG:map01230 “Biosynthesis of amino acids”. The sub regions which are grouped by dashed lines were obtained from other KEGG maps as follow: a) KEGG:map00750 “Vitamin B6 metabolism”, b) KEGG:map00730 “Thiamine metabolism”, c) KEGG:map00230 “Purine metabolism”, d) KEGG:map00240 “Pyrimidine metabolism”, e) KEGG:map00290 “Sulfur metabolism”, and f) KEGG:map00760 “Nicotinate and nicotinamide metabolism”.

TABLE S3
Bicluster #1 top 5 enriched functional pathways

| p-value | Pathway Description |
|---|---|
| 0 | Metabolic pathways |
| 0 | Biosynthesis of amino acids |
| 6.20E−15 | Biosynthesis of secondary metabolites |
| 4.90E−12 | Histidine metabolism |
| 2.91E−08 | Phenylalanine, tyrosine |
| | and tryptophan biosynthesis |

Growth phenotype data can be used not only to analyze conditional essentiality and dispensability of genes for specific environmental settings, but also to facilitate computational analysis to gain new insights into the functional organization of genes. Since about one-third of the protein-coding genes are still uncharacterized (i.e. orphan genes) even in E. coli—one of the most well-known biological systems—such analysis may be crucial to unraveling how the interplay of genetic and environmental factors orchestrates cellular-level phenotypes.

To illustrate this point, the genes in the largest bicluster found in one embodiment were examined and the function of ycdY was analyzed, which is the only orphan gene in this bicluster. This orphan gene codes for a chaperone protein that was suggested to be a redox enzyme maturation protein (REMP). No functional annotation was defined for ycdY. Surprisingly, ycdY deletion had strong effects on growth under these 10 conditions (P-value=3.33×10⁻¹⁶). In order to predict its function, the most significantly enriched GO terms in this bicluster was determined. Seventy-one out of the 79 genes (89.9%) were annotated as “organonitrogen compound biosynthetic process” whereas only 485 genes were annotated as this GO term among all the 3979 E. coli genes in this dataset, which gave a P-value of 9.57×10⁻⁵⁵. Other most significantly enriched GO terms were cellular amino acid biosynthetic process (P-value=1.37×10⁻⁴⁹), small molecule biosynthetic process (P-value=1.13×10⁻⁴⁸), cellular amino acid metabolic process (P-value=2.18×10⁻⁴³) and organonitrogen compound metabolic process (P-value=2.08×10⁻⁴²). Therefore, the analysis strongly suggests the function of ycdY to be associated with these five GO terms.

Another case study on a bicluster containing 11 genes that are essential under three dyeing chemical conditions but are dispensable under a cold shock and an antibiotic, Spectinomycin, condition also demonstrated the value of the GRACOB device and method (see FIG. 15 and Case Study No. 2).

Provided herein is a graph-based biclustering device and method that is able to determine co-fit genes from large growth phenotype profiling datasets. The GRACOB device and method are able to mine growth phenotype data. Experimental results from both a variety of synthetic datasets and three genome-scale growth phenotype datasets for E. coli, proteobacteria, and yeast demonstrated the superior performance of the GRACOB device and method over other methods.

Case Study of the Largest Bicluster Detected in One Embodiment

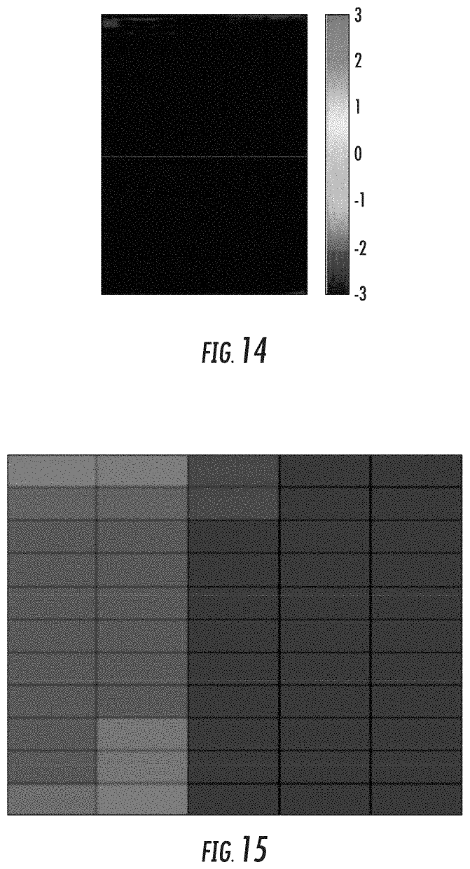

In Escherichia coli, carbon, nitrogen, and iron sources are transported and metabolized by inducible pathways. These pathways require all amino acids, purines, pyrimidines, and cofactors to be synthesized in the cell as their uptake from the medium is not adequate for the rapid utilization induced by these test conditions. The growth of the cell results from enormous various chemical reactions. These reactions require energy, vitamins, amino acids, purines, and pyrimidines. The limitation of the supply of any of these elements may impact the cellular growth. In this bicluster, the biclustered carbon, nitrogen, and Fe source stress conditions (Table S2) represent all the used variety of these types in the source data matrix. As can be seen from the heatmap in FIG. 14, all of these test conditions showed growth phenotype across all biclustered genes.

Carbon-Source Stress Conditions

Escherichia coli can grow on different types of sugars such as the listed carbon sources in the bicluster stress conditions (Table. S2). Each sugar type may go through a specific metabolic pathway where it will be broken down to intermediates (e.g. pyruvate or acetyl-CoA), which are used by other pathways to synthesize cell requirements such as energy molecular (i.e. ATP), amino acids, vitamins, nucleotides, etc. As amino acids are the building blocks of proteins, which account for 52% of the dry weight of the cell, E. coli utilizes the majority of its ATP resource in amino acids synthesis. The growth rate of a strain can be measured as a function of the carbon source. At the final stage of most sugar metabolic pathways, glucose-6-phosphate or fructose-6-phosphate will be produced. The strain that uses different carbon source as growth medium will use different enzymes for catabolism and transportation systems. However, the mutant strains which lost a key function in such specific pathway due to gene deletion, are expected to show growth phenotype in that specific growth medium but not in other mediums. For instance, glucose and acetate use different metabolic pathways. The gene ‘acs’ is involved in acetate metabolism but not in glucose, therefore, its deletion mutant is hypersensitive in acetate but not glucose. Similarly, the genes sdhA, sdhB are involved in succinate metabolic pathway but not in glucose. In agreement with that, their mutant strains showed phenotype in the succinate stress condition but not in glucose. Therefore, such genes which are specific to a stress condition and not others were not included in this bicluster where all biclustered genes show similar phenotype for all biclustered stress conditions.

Nitrogen-Source Stress Conditions

Ammonia is used by E. coli to formulate an amino group which can be utilized in the biosynthesis of most amino acids. The utilization of nitrogen source in E. coli using α-ketoglutarate (α-KG) may result in glutamate and glutamine synthesis. Glutamate is synthesized by two pathways through the combined actions of Glutamine synthetase and glutamate synthase. Glutamine synthetase (GS) catalyzes the only pathway for glutamine biosynthesis. If the concentration of ammonia is high in the growth medium, the synthesis of the enzymes utilizing it may be repressed as there are adequate nitrogen substrates in the cell. In general, the ratio between nitrogen uptake and carbon uptake may be kept constant by a regulatory network.

Fe Stress Conditions

The material used in this test was Ferrous Sulfate (FeSO₄). For the excess level stress test case, the concentration was 1 mM, and for the starvation stress test the concentration was 2 μM, while the normal cell requirement was 100 μM. The iron-sulfate clusters are essential for their metabolic role as cofactors for proteins that are involved in redox and non-redox catalysis, electron transportation, and sensing the environment conditions for oxygen and iron. In Escherichia coli, almost 40 genes are regulated by iron. In natural environments, the cell suffers iron shortage, where metal ion functions as cofactor in many of the cellular constituents such as flavoproteins. Therefore, the cell optimizes the mechanism for iron uptake and storage system. However, excess iron causes toxicity by catalyzing the formation of reactive free radicals through some reactions. Carbon and ion utilization have functional interactions between each other where many ion transport genes and several catabolic genes are subject to dual control. In E. coli, these genes are repressed by the loss of Crp, which regulates a set of genes in response to C-source, and activated by the loss of Fur, which regulates a set of genes in response to metal availability.

Biclustered Mutant Strains Overview

The majority of knocked out genes in the mutant strains in this bicluster were involved in biosynthesis pathways of 15 amino acids out of the 20 amino acids found in proteins (Table S1). The enrichment analysis of functional pathways of these genes yield similar observation (Table S3). Amino acid biosynthesis genes are dispensable in general, since the cell can obtain its needs from the environment. This is true indeed in the experimental data used in this case study. For instance: arginine, histidine, valine, and some other amino acid biosynthesis genes mutant strains showed no growth phenotype in at least 300 stress conditions out of the 324 stress conditions used in these experiments. By growth phenotype is meant the fitness value that lies an abnormal distance from other values in the same stress test condition, or the outlier value. However, in some cases these mutant strains would express growth phenotype if the available amino acids in the medium were inadequate due to rapid utilization that was triggered by external stress and a broken synthesis pathway due to the mutation. The genes found in this bicluster were the knocked out genes of strains that showed phenotype in all the biclustered stress conditions. Therefore, mutant strains of genes that show growth phenotype in part of the biclustered condition set were not included in the bicluster as per the GRACOB device and method. Therefore, the biclustered conditions represented the area of similarities among the biclustered genes at a certain level of biological function. In the following subsections some features of the mutant genes in this bicluster are highlighted:

Arginine Biosynthesis Biclustered Genes arg[A, B, C, E, G, H]

The GRACOB device and method included six genes from the arg family. These genes were distributed among four operons: argA, argCBH, argE, and argG. All of these genes play key roles in the arginine biosynthesis and showed hypersensitivity to the biclustered stress conditions. The arginine biosynthesis can be divided into two main parts: 1) biosynthesis reactions leading from glutamate to ornithine; which involve argA, B, C, D, E genes, 2) biosynthesis reactions leading from ornithine to arginine; which involve argF, I, G, H genes. argA is the structural gene of N-acetylglutamate synthase, which is the first enzyme in the arginine biosynthesis. The enzyme is feedback inhibited by arginine and regulated negatively by argR. The argECBH genes form a tight cluster within Escherichia coli genome. argCBH genes are located in a single operon, while argE is oriented in an opposite direction of the adjacent arg genes. argG transcription was shown to be activated by cAMP-CAP complex. argE is the intermediate step that produces ornithine, and argH is involved in the last step of the arginine biosynthesis pathway. Among the nine genes involved in the arginine biosynthesis only argD, argF, and argI were not included in the bicluster since they showed no growth phenotype for the biclustered conditions. The main reason for that is having another gene beside the deleted one in the mutant strain that can perform the missing function of the deleted one. For instance, each one of argF and argI genes is able to produce ornithine carbamoyltransferase which catalyzes the sixth step in the arginine biosynthesis. Therefore, if one of these two genes is mutated, its function may be complemented by the other one and no phenotype may be observed. Similarly, argD and dapC genes share common functionalities. argD encodes acetylornithine aminotrans-ferase (NAcOATase), and dapC encodes L-diaminopimelate: α-ketoglutarate aminotransferase (DapATase). The NAcOATase enzyme performs similar reaction to that of DapATase, catalyzing the N-acetylornithine-dependent transamination of α-ketoglutarate.

Chorismate Biosynthesis Biclustered Genes aro[A, B, C, E]

Chorismate is an intermediate in biosynthesis of aromatic amino acids: i.e. phenylalanine, tryptophan, and tyrosine. aroA gene encodes 5-enolpyruvylshikimate-3-phosphate synthase enzyme (EPSP synthase) which catalyze a reaction in the biosynthetic pathway leading to chorismate. aroA gene is part of an operon that include serC gene which is involved in the serine biosynthesis. Serine and chorismate are precursors of enterochelin which is a high affinity siderophore that is required for iron uptake. The serC-aroA operon was found positively regulated by cAMP. The Chorismate biosynthesis pathway include the genes aroB, D, E, (K, L), A, C in that order. Only aroD, aroK, and aroL were missing from the bicluster. The gene aroD was not included in the final experimental data by the source, while the genes aroK and aroL both share similar functionality in the pathway as Shikimate kinase.

Cysteine Biosynthesis Biclustered Genes cys[B, C, D, G, I, J, K, Q]

Sulfur is a fundamental atom in cysteine and methionine amino acids and number of various coenzymes and cofactors. The cysteine biosynthesis is the major pathway of sulfur assimilation. The general cysteine biosynthesis pathway involves more than 15 genes from Cysteine family and can be divided into two main pathways beside the sulfate transportation function which involves cysPTWA operon. The pathways are: 1) the assimilation of sulfur from sulfate, 2) the biosynthesis of cysteine from serine, which is also a precursor for methionine and a number of other components. In Escherichia coli, the genes from the first pathway are organized into three operons: cysDNC, cysJIH, and cysG, while the genes from the second pathway are cysE, cysK, and cysM. These pathways are feedback regulated at different levels by various products of the pathways. In addition, cysB and cysQ plays important regulatory role in the biosynthesis of cysteine. CysB controls the transport of sulfate and cysteine for sulfate reduction and its assimilation into cysteine. The transcription of most cys genes is positively regulated by the protein product of cysB. CysQ is responsible for regulating the sulfate assimilation pathway by influencing levels of intermediates in the cell, and it was shown to be required during aerobic growth in E. coli to help control the level of 3′-phosphoadenosine 5′-phosphosulfate (PAPS) in cysteine biosynthesis. PAPS is formulated by adenosine phosphosulfate (APS) kinase, which is encoded by cysC. APS formulation requires two proteins, cysD and cysN. Besides the role in cysteine pathway, APS is also involved in another sulfur cycle that transform APS to sulfite and AMP by an APS reductase.

In this bicluster there were 8 cys genes out of 12 genes from the two pathways. Three of the four remaining genes, i.e. cysE, cysH, and cysN, were not included in this bicluster either due to having missing or less significant fitness value for some test conditions. However, all these 11 cys genes were listed together in another bicluster returned by the GRACOB device and method where all the genes had similar fitness values for the biclustered test conditions. Only cysM was not found in any bicluster mainly due to showing no phenotype in stress condition test set of any bicluster resulted from the used thresholds in the GRACOB device and method run. However, this gene shares the same functionality with cysK, both genes convert O-acetyl-L-serine (OAS) into cysteine and acetate. In addition, both genes match in 43% of their amino acid sequence. Therefore, strains lacking either of these two genes are cysteine prototrophs. The sulfate transportation cys genes, i.e. cysPTWA, were not included in the bicluster too, mainly due to showing no phenotype in some of the biclustered test conditions, except cysT was missing from source. Interestingly, cysA and cysW were known to be heat-shock genes and were biclustered with high temperature stress conditions in another bicluster returned by the GRACOB device and method.

Histidine Biosynthesis Biclustered Genes his[A, B, C, D, F, G, H, I]

Histidine biosynthesis pathway consists of a single operon, hisGDCBHAFI, which encodes the eight enzymes involved in the pathway. There are ten steps in this pathway, following is a brief: ATP phosphoribosyltransferase enzyme catalyzes the first step in the pathway. The enzyme is encoded by hisG gene. The enzyme activity is inhibited by a number of interrelated methods such as feedback inhibition by histidine, and also can be competitively inhibited by ADP and AMP. The second and third steps are performed by a bifunctional enzyme encoded by hisI. The enzyme first catalyzes phosphoribosyl-ATP pyrophosphohydrolase then phosphoribosyl-AMP-cyclohydrolase. The forth step is carried out by hisA which catalyzes a reaction known as Amadori rearrangement. Then hisF and hisH work together to catalyze a reaction which uses glutamine to produce 5-aminoimidazole-4-carboxamide ribonucleotide and imidazoleglycerol phosphate. The bifunctional enzyme encoded by hisB will catalyze the sixth and eighth steps. In the sixth step, hisB enzyme will dehydrate D-erythro-imidazole-glycerol-phosphate to yield imidazole acetol-phosphate. Then Histidinol-phosphate aminotransferase enzyme, hisC, will help convert imidazole acetol-phosphate to histidinol-phosphate. Next, hisB will come in the picture again to convert L-histidinol-phosphate into histidinol. The final two steps are handled by hisD which will catalyze the dehydrogenation of histidinol to produce histidinal and then the dehydrogenation of histidinal to yield L-histidine. All of these genes showed growth phenotype and were included in this bicluster.

Valine, Isoleucine and Leucine Biosynthesis Biclustered Genes ilv[A, C, D, E], and leuB

Valine, isoleucine, and leucine are synthesized through the branched-chain amino acids (BCAAs) pathway. Most of the enzymes catalyzing the reactions in this pathway are common in the synthesis of these three amino acids. The first enzyme in the BCAAs pathway is Acetohydroxyacid Synthase (AHAS). The AHAS enzyme catalyzes decarboxylation of pyruvate. There are three isoenzymes each of which can perform the function of AHAS. They are encoded by ilvBN, ilvGM, and ilvIH. The second step is performed by Acetohydroxyacid Isomeroreductase (AHAIR), encoded by ilvC. AHAIR catalyzes the conversion of acetohydroxyacids into dihydroxyacids. The third step in BCAAs pathway is carried out by Dihydroxyacid Dehydratase (DHAD), encoded by ilvD. The enzyme can perform two parallel reactions the first converts 2,3-dihydroxyisovalerate into 2-keto-isovalerate which is a precursor for isoleucine and the second converts 2,3-dihydroxy-3-methylvalerate to 2-keto-3-methyl-valerate which is a precursor for valine and leucine. The last reaction in the BCAAs pathway is catalyzed by the common enzyme Transaminases (TAs), encoded by ilvE. Only isoleucine biosynthesis requires an extra enzyme to catalyze the reaction of converting L-threonine to 2-ketobutyrate which is a precursor for isoleucine and an inducer for AHAS. This enzyme is Threonine Deaminase (TD), encoded by ilvA. Leucine synthesis requires three more enzymes to produce the required precursor for TA to synthesis leucine. They are ordered as follows: Isopropylmalate synthase (leuA), Isopropylmalate dehydratase (leuCD), and Isopropylmalate dehydrogenase (leuB).

The bicluster contained 5 genes out of the 9 genes that are not coding for the AHAS isoenzymes. As mentioned earlier, isoenzyme single gene mutation may not be expected to show growth phenotype since other gene(s) may complement the missing one. The 4 missing genes from the bicluster were lacking the fitness value of one of the stress test conditions in the bicluster, however, all the 9 genes were biclustered together in another bicluster returned by the GRACOB device and method which did not include the test condition with the missing value.

Lysine Biosynthesis Biclustered Genes lys[A, R]

Lysine is synthesized from aspartate through diaminopimelic acid (DAP) pathway in bacteria. There are four different DAP pathways in bacteria: the acetylase, aminotransferase, dehydrogenase, and succinylase pathways. These pathways convert aspartate to tetrahydrodipicolinate using common steps, however, the steps to synthesis meso-diaminopimelate, which is a precursor for lysine, are different. The succinylase dependent pathway is known to exist in eubacteria, e.g. E. coli. The first step in the DAP pathway can be catalyzed by any of the isoenzymes encoded by lysC, metL, and thrA.

This step is common among diaminopimelate, isolecucine, lysine, methionine, and threonine biosynthesis pathways. The genes involved in the succinylase pathway are dapD, dapC, dapE, and dapF. Diaminopimelate decarboxylase enzyme, encoded by lysA, catalyzes the last step in lysine biosynthesis pathway. The lysA gene requires an activator, lysR, for its expression. lysA and lysR may be expected to express lysine auxotrophy phenotype. Only these two lysine genes were biclustered.

Methionine Biosynthesis Biclustered Genes met[A, B, C, E, F, R]

In Escherichia coli, methionine is synthesized from aspartate amino acid. As shown previously, aspartate is a key precursor for a number of amino acids such as lysine and methionine. The isoenzymes catalyzing aspartate phosphorylation to yield aspartyl-phosphate are encoded by lysC, metL, and thrA. The aspartyl-phosphate is converted to aspartate-semialdehyde by aspartate-semialdehyde dehydrogenase which is encoded by asd. Then aspartate-semialdehyde is reduced to homoserine by homoserine dehydrogenase. In E. coli there are two isoenzymes can catalyze this reaction metL and thrA. Then, homoserine transsuccinylase, encoded by metA, catalyzes the synthesis of O-succinyl-homoserine from succinyl-CoA and homoserine. Next, metB use O-succinyl-homoserine and cysteine to produce γ-cystathionine. The metC gene, encoding for cystathionine-β-lyase, converts γ-cystathionine to ammonia, homocysteine, and pyruvate. The final step in this pathway can be catalyzed by two different enzymes, the vitamin B12-dependent methionine synthase, encoded by metH, and the vitamin B12-independent methionine synthase, encoded by metE. The metE mutant would require methionine or vitamin B12 for growth. The methionine can be repressed by metJ and activated by metR. The gene metF encode for methylene-tetrahydrofolate (THF) reductase which catalyze a reduction from CH2-THF to CH3-THF. The metF mutant would lead to methionine limitation. The s-adenosylmethionine (SAM) is a key precursor for a number of important metabolites. The gene metK encodes for SAM synthetase which catalyze the SAM synthesis. Therefore, metK gene is known to be essential in E. coli, and its deletion mutant was not included in the experimental data by the source. All the key genes in methionine biosynthesis pathway were biclustered together in this bicluster.

Proline Biosynthesis Biclustered Genes pro[A, B, C]

In Escherichia coli proline is synthesized from glutamate. There are three enzymes catalyzing the reactions in this process: γ-glutamyl kinase (GK), γ-glutamyl phosphate reductase (GPR), and Δ-pyrroline-5-carboxylate reductase (P5CR), encoded by genes proB, proA and proC, respectively. GK forms a complex with GPR to catalyze the reaction that convert glutamate to γ-glutamyl phosphate. The γ-glutamyl phosphate is converted nonenzymatically to Δ1-pyrroline-5-carboxylate, which is then reduced to proline by P5CR. All these genes were included in this bicluster as they expressed growth limitation under all the biclusters stress conditions.

Serine and Glycine Biosynthesis Biclustered Genes ser[A, C], and glyA

The serine biosynthesis pathway consists of three steps. First, the 3-phosphoglycerate dehydrogenase enzyme, encoded by serA, produces 3-phosphohydroxypyruvate through an NAD dependent reaction. Then, phosphoserine aminotransferase, encoded by serC, catalyzes the second reaction to obtain 3-phosphoserine by amino transfer from 1-glutamate. The gene serB encoded enzyme, phosphoserine phosphatase, catalyzes the last reaction to produce serine. Finally, serine hydroxymethyltransferase (glyA), convert serine to glycine. Only, serB was not included in this bicluster due to missing fitness values for 2 of the 10 stress test conditions. However, all the 3 genes were biclustered together in another bicluster returned by the GRACOB device and method which only included the 8 conditions.

Threonine Biosynthesis Biclustered Genes thr[A]

There are three threonine genes involved in the threonine biosynthesis contained in the operon thrABC. The gene thrA plays two roles in the pathway the first as aspartate kinases I, and the second is homoserine dehydrogenase. The homoserine kinase, encoded by thrB, catalyzes the phosphorylation of homoserine to homoserine phosphate. The final step in the threonine biosynthesis is carried out by threonine synthase, encoded by thrC. The genes thrB and thrC were missing from this bicluster due to missing fitness values for a test condition, however, all the three genes showed growth phenotype for the remaining 9 test conditions and were biclustered together in another bicluster.

Tryptophan Biosynthesis Biclustered Genes trp[A, B]

The biosynthesis of tryptophan from chorismate requires five enzymes in following order: 1) an-thranilate synthase, which is a dual components that are encoded by trpE, trpD; 2) phosphoribosyl-anthranilate transferase, encoded by trpD; 3) N-phosphoribosyl anthranilate isomerase, encoded by trpC; 4) indole glycerol phosphate synthase, encoded by trpC; 5) tryptophan synthase, which is a heterotetramer formed from two protein components encoded by trpA and trpB. These genes in E. coli are localized in one operon trpEDCBA. The operon is promoted by trpL and can be repressed by trpR. For the test conditions of this bicluster only trpA and trpB showed growth phenotype for all the conditions. The other available genes, trpE, trpD, trpC, trpR, showed normal growth fitness values in most of the test conditions.

NAD Biosynthesis Biclustered Genes nad[A, B, C]

The nicotinamide adenine dinucleotides (NAD) and its derivatives (NADH, NADP, and NADPH) are essential cofactors in all living systems. They function in many anabolic and catabolic reactions which can be found in different pathways. Amino acid biosynthesis pathways in E. coli utilize these coenzymes in many reactions such as the NADPH-dependent reduction reaction catalyzed by argC in arginine pathway, the reaction catalyzed by aroB and aroE in the aromatic amino acids pathway would essentially need NAD+ for their catalytic activities. Many genes in cysteine, histidine, isoleucine, valine, and methionine biosynthesis pathways are using these coenzymes. Following enzymes are required for the biosynthesis of NAD and its derivatives: aspartate oxidase (nadB), quinolinate synthase (nadA), quinolinate phos-phoribosyltransferase (nadC), nicotinic acid mononucleotide adenylyltransferase (nadD), NAD synthetase (nadE), and NAD kinase (nadF and nadG). Only nadA, nadB, and nadC deletion mutant were included in the experimental data and they showed growth phenotype, the other nad gene mutation strains were not available from source. Thus, nadA, nadB, and nadC were included in this bicluster.

Carbamoylphosphate Biosynthesis Biclustered Genes car[A, B]

The carAB operon in Escherichia coli encode the two subunits of carbamoylphosphate syn-thetase. The carbamoylphosphate is a common precursor of arginine, and pyrimidine pathways. The operon synthesizes the carbamoyl phosphate from glutamine. This pathway can be regulated by arginine, UMP, IMP, and ornithine. Mutants on the carAB operon would lead to uracil and arginine double requirements phenotype.

Pyrimidines Biosynthesis Biclustered Genes pyr[C, D, E]

Pyrimidines derivatives such as uracil, cytosine, and thymine are known building blocks of DNA and/or RNA. Other derivatives such as OMP, UMP, UDP, etc. play key roles in cell signaling and regulation. The pyrimidine genes pyrB, I, C, D, E, F, H, and G are involved in the pyrimidines biosynthesis pathway in that order. The genes pyrI and pyrF showed no growth phenotype in all reported tests. The gene pyrB showed similar growth phenotype in all tests to the biclustered genes except for one stress test condition. These 4 gene, pyrBCDE, were biclustered together in another bicluster return by the GRACOB device and method. The genes pyrG and pyrH were not included in the bicluster due to their deletion mutant of being missing from source.

Purines Biosynthesis Biclustered Genes pur[A, C, D, E, H, K, L, M]

Adenine and guanine are purines which are found in DNA and RNA. The purine genes that take roles in the purines pathway are purF, D, N, T, L, M, T, G, I, E, K, C, B, H, J, and A. All of these genes were included in the bicluster except purG, purl, purB, and purJ were not included due to being missing from source data, and the isoenzyme genes purN and purT. These isoenzyme genes are catalyzing the same step in the synthesis pathway. Therefore, a single gene mutation in any of these two genes was not expected to break the purines synthesis nor show a growth phenotype.

Thiamine Biosynthesis Biclustered Genes thi[C, D, E, F, G]

Thiamine, vitamin B1, is synthesized from an intermediate product of purine biosynthesis pathway. The derivatives of thiamine, e.g. thiamine pyrophosphate (TPP), are involved in many cellular reactions as coenzymes such as in the valine biosynthesis and glycolaldehyde transferase. The thiamine genes involved in the thiamine biosynthesis pathway are thiF, I, M, G, H, C, D, L, and K. The genes thiBPQ are coding for thiamine transport system. Only thiL was missing from the data source. The genes thiH, I, S were not included in this bi-cluster due to showing no phenotype for some of the stress conditions, however, all the available eight genes were biclustered together in another result returned by the GRACOB device and method. The mutant strains of genes thiM, K, B, P, and Q were expected not to show thiamine requirement phenotype since they participate in thiamine transport or salvage pathway.

Pyridoxine Biclustered Genes pdx[A, H, J]

Pyridoxine, vitamin B6, is a precursor of pyridoxal phosphate, which is an essential coenzyme for many reactions in the amino acid metabolism pathway. The genes involved in the pyridoxine biosynthesis are tktA, tktB, talA, talB, gapB, pdxB, serC (pdxC), pdxA, pdxJ and pdxH. The isoenzymes (tktA and tktB), and (talA and talB) showed no growth phenotype as expected. The gapB null mutant was not included in the source data. The gene pdxB was not included in this bicluster due to no growth phenotype was shown for some of the biclusters stress conditions, however, pdxB was biclustered with the other pdx genes in another bicluster returned by the GRACOB device and method.

Non-Biclustered Amino Acids Genes

The strains of mutant genes which are involved in the biosynthesis of five of the protein coding amino acids were not included in this bicluster. The main reason for that as will be shown below is due to having multiple pathways to synthesis these amino acids where a single mutation is less likely to show a growth phenotype. In the following subsections these amino acids biosynthesis pathways will be discussed:

Aspartic Acid and Asparagine

There are two separate reactions each of which can synthesis aspartic acid. The genes coding for the enzymes in these reactions are aspC and tyrB. Aspartate requirement can be caused by a double mutation in aspC and tyrB. Similarly for asparagine, it has two different pathways for biosynthesis each of them can provide adequate supply of asparagine. The genes involved in these pathway are asnA and asnB.

Glutamine

Besides its role as a protein building block, glutamine plays a key role in the amino acid biosynthesis by supplying the pathways with amide groups in transamination or transamidation reactions. There are two genes involved in glutamine synthesis, glnA and glnE. The glnA showed phenotype for all conditions in this bicluster except for 3 of them where the fitness value missing from source. The other gene, glnE, was not available in the source data at all.

Glutamate

In Escherichia coli, there are number of pathways for glutamate synthesis. For instance, glutamate synthase (gltB and gltD), and glutamate dehydrogenase (gdhA) enzymes can synthesize glutamate. Also, the arginine succinyltransferase pathway, encoded by genes in the operon astCADBE, produces glutamate at its final step. The gene asnB catalyzes a reaction which yields glutamate from glutamine and aspartate. None of these genes showed growth phenotype in the biclustered test conditions.

Alanine

Alanine can be synthesized from pyruvate through two different pathways each of which can provide the cell with adequate supply of alanine. An alanine auxotroph strain may not have ever been isolated, which indicate existence of multiple alanine synthesis pathways.

Case Study No. 2: Case Study of a Mixed-Color Bicluster Detected by the GRACOB Device and Method

This case study contained a bicluster of 11 genes that are essential under three dyeing chemical conditions but are dispensable under a cold shock and an antibiotic, Spectinomycin, condition. FIG. 15 illustrates a sample bicluster of size 11×5 with mixed colors that illustrate a grouping of genes based on both conditional essentiality and dispensability criteria. The 11 genes are listed in Table S4 and the 5 conditions are listed in Table S5, below.

TABLE S4
Mutant genes from bicluster #2
Δ genes

| | | | | | |
|---|---|---|---|---|---|
| lipA | nuoA | nuoE | nuoG | nuoH | nuoJ |
| nuoK | nuoM | nuoN | ubiF | yfjG | |

TABLE S5
Bicluster #2 stress test conditions

| Stress test label | Test family |
|---|---|
| Temperature-20 C. | Cold shock |
| Spectinomycin-4.0 | Aminoglycoside |
| Acriflavine-10 | Dye |
| Ethidium Bromide-2 | Dye |
| Pyocyanin-10.0 | Phenazine |

TABLE S6
Bicluster #2 top 5 enriched functional pathways

| p-value | Pathway Description |
|---|---|
| 5.11E−15 | Oxidative phosphorylation |
| 1.26E−13 | Nitrogen metabolism |
| 5.99E−08 | Metabolic pathways |
| 0.008273 | Lipoic acid metabolism |
| 0.038070 | Ubiquinone and other terpenoid-quinone biosynthesis |

This bicluster contained mutant strains of 8 Nuo genes which are members of a single operon. The 8 genes code for enzymes that bind together to form a compound named “NADH dehydrogenase I” which couples the electron transfer from NADH to ubiquinone with a proton translocation.

The mutant strains in this bicluster showed resistance phenotype to 2 distinct stress test conditions and showed growth inhibition for the other 3 stress conditions. The first resisted stress was a “cold shock,” in which the mutant culture was exposed to a dramatic reduction of the temperature, i.e. the culture temperature was reduced from 37° C. to 20° C. in this specific stress test condition. Such a change should trigger the cold shock response system of the E. coli cell. None of the biclustered knocked out genes was a member of this system, and therefore all the mutant strains were able to resist the condition. The other resisted test was the antibiotic “Spectinomycin,” which inhibits protein synthesis on the E. coli ribosomes by impacting its initial selection and proof-reading steps. In agreement with the observed phenotype in this bicluster, the impact of “Spectinomycin” on NADH was measured in a previous study, which concluded no effect of this antibiotic on the level of NADH.

The growth inhibition conditions were all dyeing chemicals. They also shared a behavior of inducing the intracellular production of the toxic superoxide. This oxidative stress was shown to deplete NADH in wildtype and almost all its genes, including the genes found in this bicluster, were significantly activated when exposed to the stress. Therefore, these genes are essential under these conditions for the cell survival.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A device for detecting co-fit genes, the device comprising a processor and a memory storing computer instructions that, when executed by the processor, cause the device to:
transform genome-wide growth-phenotype data using a cumulative distribution function into transformed phenotype data disposed in a plurality of rows and columns;
sort the transformed phenotype data disposed in the plurality of columns independently of each column of the plurality of columns while retaining an original row index associated with each transformed phenotype data;
create a node for each set of consecutive rows in the plurality of rows;
create an edge between a pair of nodes in response to the pair of nodes being from different data columns sharing a number of consecutive rows over a row threshold;
delete any nodes having a number of consecutive rows under a column threshold;
determine maximal cliques from any remaining pairs of nodes; and
extract biclusters from the cliques to detect the co-fit genes.

2. The device of claim 1, wherein the plurality of columns represents a plurality of stress conditions.

3. The device of claim 1, wherein the plurality of rows represents a plurality of strains.

4. The device of claim 1, wherein the nodes are created for each set of consecutive rows in the plurality of rows such that the range of the transformed phenotype data in each consecutive row of the set of consecutive rows does not exceed a range threshold.

5. The device of claim 2, wherein the range threshold is a numerical range in which the transformed phenotype data of each consecutive row of the set of consecutive rows must fall.

6. The device of claim 3, wherein range threshold is about 0.01 to about 0.10.

7. The device of claim 1, wherein the transformed phenotype data is sorted in ascending order.

8. The device of claim 1, wherein the memory storing computer instructions that, when executed by the processor, cause the device to repeat creation of an edge and deletion of any nodes.

9. The device of claim 1, wherein the row threshold represents a number of strains or genes in each bicluster.

10. The device of claim 1, wherein the column threshold represents a number of stress conditions imposed on a strain or gene in the bicluster.

11. A method of detecting co-fit genes, the method comprising:
transforming genome-wide growth-phenotype data using a cumulative distribution function into transformed phenotype data disposed in a plurality of rows and columns;
sorting the transformed phenotype data disposed in the plurality of columns independently of each column of the plurality of columns while retaining an original row index associated with each transformed phenotype data;
creating a node for each set of consecutive rows in the plurality of rows;
creating an edge between a pair of nodes in response to the pair of nodes being from different data columns sharing a number of consecutive rows over a row threshold;
deleting any nodes having a number of consecutive rows under a column threshold;
determining maximal cliques from any remaining pairs of nodes; and
extracting biclusters from the cliques to detect the co-fit genes.

12. The device of claim 1, wherein the plurality of columns represents a plurality of stress conditions.

13. The device of claim 1, wherein the plurality of rows represents a plurality of strains.

14. The method of claim 11, wherein the nodes are created for each set of consecutive rows in the plurality of rows such that the range of the transformed phenotype data in each consecutive row of the set of consecutive rows does not exceed a range threshold.

15. The method of claim 14, wherein range threshold is a numerical range in which the transformed phenotype data of each consecutive row of the set of consecutive rows must fall.

16. The method of claim 14, wherein range threshold is about 0.01 to about 0.10.

17. The method of claim 11, wherein the transformed phenotype data is sorted in ascending order.

18. The method of claim 11, further comprising repeating the creation of edges and deletion of any nodes.

19. The method of claim 11, wherein the row threshold represents a number of strains or genes in each bicluster.

20. The method of claim 11, wherein the column threshold represents a number of stress conditions imposed on a strain or gene in the bicluster.
